(19) United States
(12) Patent Application Publication
(10) Pub. No.: US 2025/0352535 A1
(43) Pub. Date: Nov. 20, 2025

(54) MDM2 DEGRADERS AND USES THEREOF

(71) Applicant: Kymera Therapeutics, Inc., Watertown, MA (US)

(72) Inventors: Jeffrey Davis, Watertown, MA (US); Ashwin Gollerkeri, Watertown, MA (US); Reginald Ewesuedo, Watertown, MA (US); Sagar Agarwal, Watertown, MA (US); Chris Ho, Watertown, MA (US); Rachelle Perea, Watertown, MA (US); Patrick Henrick, Watertown, MA (US); Michele Mayo, Watertown, MA (US); Jianfeng Qi, Watertown, MA (US); Alice McDonald, Watertown, MA (US); Yogesh Chutake, Watertown, MA (US)

(21) Appl. No.: 18/872,432

(22) PCT Filed: Jun. 6, 2023
(86) PCT No.: PCT/US2023/024543

§ 371 Date: Dec. 6, 2024

Related U.S. Application Data
(60) Provisional application No. 63/349,350, filed on Jun. 6, 2022.
(60) Provisional application No. 63/375,822, filed on Sep. 15, 2022.
(60) Provisional application No. 63/384,044, filed on Nov. 16, 2022.
(60) Provisional application No. 63/387,651, filed on Dec. 15, 2022.
(60) Provisional application No. 63/484,259, filed on Feb. 10, 2023.

(51) Int. Cl. A61K 31/4545; A61K 9/00; A61K 47/10; A61K 47/26; A61P 35/00; A61P 35/02

(52) U.S. Cl. CPC A61K 31/4545; A61K 9/0019; A61K 47/10; A61K 47/26; A61P 35/00; A61P 35/02

(57) ABSTRACT

The present invention relates to MDM2 degraders, their liquid formulations, and methods of use thereof for treating cancer.

### CROSS-REFERENCE TO RELATED APPLICATIONS

This application claims the benefit of priority to U.S. Provisional Appl. No. 63/349,350, filed Jun. 6, 2022, U.S. Provisional Appl. No. 63/375,822, filed Sep. 15, 2022, U.S. Provisional Appl. No. 63/384,044, filed Nov. 16, 2022, U.S. Provisional Appl. No. 63/387,651, filed Dec. 15, 2022, and U.S. Provisional Appl. No. 63/484,259, filed Feb. 10, 2023, the contents of each of which is herein incorporated by reference.

### TECHNICAL FIELD OF THE INVENTION

The present invention relates to formulations and dosages forms of MDM2 degrader (3′R,4′S,5′R)-6″-chloro-4′-(3-chloro-2-fluorophenyl)-N-((1R,4R)-4-(4-(1-(2,6-dioxopiperidin-3-yl)-3-methyl-2-oxo-2,3-dihydro-1H-benzo[d]imidazol-5-yl)piperidine-1-carbonyl)cyclohexyl)-2″-oxodispiro[cyclohexane-1,2′-pyrrolidine-3′,3″-indoline]-5′-carboxamide (Compound A), and methods of use thereof.

### BACKGROUND OF THE INVENTION

The murine double minute 2 (MDM2) oncoprotein is a key E3 ubiquitin ligase that degrades the tumor-suppressor p53. Reversible small molecule inhibitors (SMIs) of the MDM2/p53 interaction have been developed to stabilize p53 and to induce apoptosis in wildtype p53 tumors. However, MDM2 SMIs induce a p53/MDM2 feedback loop, resulting in upregulation of MDM2 protein levels and p53 pathway inhibition thus drastically limiting their biological activity and clinical application. MDM2 targeted protein degradation suppresses p53-dependent MDM2 protein feedback upregulation and is therefore expected to lead to a superior response compared to MDM2 SMIs.

A need exists to develop formulations for MDM2 degraders for use in cancer therapy.

### SUMMARY OF THE INVENTION

It has been found that a MDM2 degrader, and its salts, formulations and unit dosage forms, as described herein, have certain advantages in treating solid cancers and hematological malignancies, wherein the MDM2 degrader is (3′R,4′S,5′R)-6″-chloro-4′-(3-chloro-2-fluorophenyl)-N-((1R,4R)-4-(4-(1-(2,6-dioxopiperidin-3-yl)-3-methyl-2-oxo-2,3-dihydro-1H-benzo[d]imidazol-5-yl)piperidine-1-carbonyl)cyclohexyl)-2″-oxodispiro[cyclohexane-1,2′-pyrrolidine-3′,3″-indoline]-5′-carboxamide (Compound A).

Accordingly, in one aspect, the present disclosure provides a liquid formulation or unit dosage form comprising Compound A, or a pharmaceutically acceptable salt thereof, and a pharmaceutically acceptable excipient and/or carrier.

In another aspect, the present invention provides a method for treating a solid cancer or hematological malignancy in a patient, comprising administering to the patient a therapeutically effective amount of Compound A, or a pharmaceutically acceptable salt thereof, or a liquid formulation described herein. In some embodiments, the solid cancer or hematological malignancy is selected from acute lymphoblastic leukemia (ALL), chronic lymphocytic leukemia (CLL), large granular lymphocytic leukemia (LGL-L), B-cell prolymphocytic leukemia, acute myeloid leukemia (AML), Burkitt lymphoma/leukemia, primary effusion lymphoma, peripheral T-cell lymphoma (PTCL), cutaneous T-cell lymphoma (CTCL), diffuse large B-cell lymphoma (DLBCL), advanced B-cell diffuse large B-cell lymphoma (ABC DLBCL), intravascular large B-cell lymphoma, lymphoplasmacytic lymphoma, Waldenström's macroglobulinemia (WM), splenic marginal zone lymphoma, multiple myeloma, plasmacytoma, uveal melanoma, myelodysplastic syndrome (MDS), or myelodysplastic/myeloproliferative neoplasms (MDS/MPN). In certain embodiments, the solid cancer or hematological malignancy is a relapsed and/or refractory (R/R) solid cancer or hematological malignancy. In some embodiments, the patient receiving Compound A or a pharmaceutically acceptable salt thereof to treat a solid cancer or hematological malignancy has received at least two prior therapies. In some embodiments, the patient is a human.

These and other aspects of this disclosure will be apparent upon reference to the following detailed description. To this end, various references are set forth herein which describe in more detail certain background information and procedures and are each hereby incorporated by reference in their entirety.

### BRIEF DESCRIPTION OF THE DRAWINGS

FIG. 1 show a flow diagram of the liquid formulation manufacturing process.

FIG. 2 shows study schema of the dose escalation with MTD/RP2D confirmation (Phase 1a) and dose expansion (Phase 1b).

FIGS. 3A and 3B shows that Compound A (1 mg/kg, Q3W) achieves tumor regression in a CTG-2227 AML patient-derived xenograft (PDX) model and partial responses in CTG-2240 and CTG-2700 AML PDX models.

FIGS. 4A and 4B shows the combinatorial benefit of Compound A with venetoclax and midostaurin in MOLM-13 cell line.

FIG. 5 shows the significant combinatorial benefit of Compound A with standard of care in AML in vivo model.

FIG. 6 shows that Compound A is active across multiple heme indications in vitro with AML, T cell lymphomas, mantle cell lymphoma, and DLBCL being the most sensitive.

FIG. 7 shows that Compound A is highly active in p53^{WT} ABC-subtype DLBCL. Compound A was highly active in OCI-LY10 p53^{WT} ABC-subtype DLBCL xenograft model (A) but not TMD8 p53^{MUT} ABC-subtype DLBCL xenograft model (B).

FIG. 8 shows that a single dose of Compound A drives sustained tumor regression and is superior to MDM2/p53 small molecule inhibitors (SMIs).

FIG. 9 shows that Compound A potently degrades MDM2 and upregulates the p53 pathway in tumors.

FIG. 10 shows a single dose of Compound A leads to robust activation of the p53 pathway and apoptosis in ALL while exposure-matched weekly dosing and SMIs do not.

FIG. 11 shows a single dose of Compound A leads to more robust activation of the p53 pathway and apoptosis than exposure-matched weekly dosing and SMIs in AML.

FIG. 12 shows that exposures are required for tumor regression are associated with induction of apoptotic markers.

### DETAILED DESCRIPTION OF CERTAIN EMBODIMENTS

### 1. General Description of Certain Embodiments of the Invention

Compound A is a highly potent heterobifunctional small molecule therapeutic agents targeting MDM2 to mediate the selective degradation of MDM2 protein. Compound A displays superior activity compared to SMIs of MDM2 in wildtype p53 cell lines and xenograft models. For instance in acute lymphoblastic leukemia (ALL) cell line RS4;11, Compound A can overcome the p53-dependent upregulation of MDM2 protein levels as seen for reversible SMIs. Short 2 hour exposures of Compound A can more potently stabilize p53 than SMIs. In addition, washout experiments in these cells showed that a pulsed dose of Compound A can lead to apoptosis mediated through p53 target genes. The superior MDM2/p53 pathway inhibition and induction of apoptosis by Compound A translates into a >200-fold stronger cell growth inhibition, compared to SMIs, across a panel of solid and hematological tumor cell lines. In some embodiments, provided herein is a treatment of adult patients with solid cancers or hematological malignances who have received at least one prior therapy. Compound A of the current invention is provided by oral and intravenous administration at the doses and schedules described herein.

Accordingly, in some embodiments, the present disclosure provides a method for treating a relapsed and/or refractory solid cancer or hematological malignancy. In some embodiments, the present disclosure provides a method for treating a relapsed and/or refractory acute lymphoblastic leukemia (ALL) or acute myeloid leukemia (AML) in a patient, comprising administering to the patient a therapeutically effective amount of Compound A, or a pharmaceutically acceptable salt thereof, or a liquid formulation thereof as described herein.

In some embodiments, the present disclosure provides a liquid formulation, which comprises Compound A, or a pharmaceutically acceptable salt thereof, and a pharmaceutically acceptable excipient and/or carrier. In some embodiments, the present disclosure provides a unit dosage form, which comprises Compound A, or a pharmaceutically acceptable salt thereof, and a pharmaceutically acceptable excipient and/or carrier.

In the following disclosure, certain specific details are set forth in order to provide a thorough understanding of various embodiments. However, one skilled in the art will understand that the methods and uses described herein may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Reference throughout this specification to “one embodiment” or “an embodiment” means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

### 2. Definitions

As used in the specification and appended claims, unless specified to the contrary, the following terms and abbreviations have the following meanings.

As used herein, the term “about” refers to within 20% of a given value. In some embodiments, the term “about” refers to within 20%, 19%, 18%, 17%, 16%, 15%, 14%, 13%, 12%, 11%, 10%, 9%, 8%, 7%, 6%, 5%, 4%, 3%, 2%, or 1% of a given value.

As used herein, the term “BCL-2 inhibitor” includes, but is not limited to compounds having inhibitory activity against B-cell lymphoma 2 protein (BCL-2), including but not limited to ABT-199, ABT-731, ABT-737, apogossypol, Ascenta's pan-BCL-2 inhibitors, curcumin (and analogs thereof), dual Bcl-2/Bcl-xL inhibitors (Infinity Pharmaceuticals/Novartis Pharmaceuticals), Genasense (G3139), HA14-1 (and analogs thereof; see WO 2008/118802, US 2010/0197686), navitoclax (and analogs thereof, see U.S. Pat. No. 7,390,799), NH-1 (Shenayng Pharmaceutical University), obatoclax (and analogs thereof, see WO 2004/106328, US 2005/0014802), S-001 (Gloria Pharmaceuticals), TW series compounds (Univ. of Michigan), and venetoclax. In some embodiments the BCL-2 inhibitor is a small molecule therapeutic. In some embodiments the BCL-2 inhibitor is a peptidomimetic.

As used herein, the term “Compound A” refers to (3′R,4′S,5′R)-6″-chloro-4′-(3-chloro-2-fluorophenyl)-N-((1R,4R)-4-(4-(1-(2,6-dioxopiperidin-3-yl)-3-methyl-2-oxo-2,3-dihydro-1H-benzo[d]imidazol-5-yl)piperidine-1-carbonyl)cyclohexyl)-2″-oxodispiro[cyclohexane-1,2′-pyrrolidine-3′,3″-indoline]-5′-carboxamide having the formula:

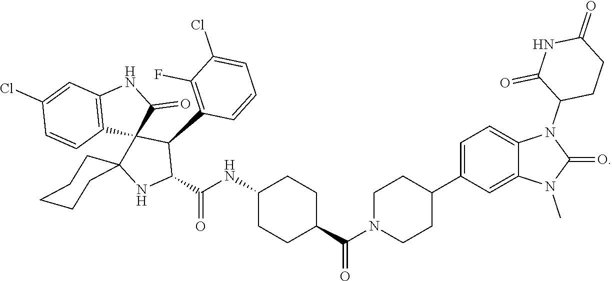 [SMILES: Cn1c(=O)n(C2CCC(=O)NC2=O)c2ccc(C3CCN(C(=O)[C@H]4CC[C@H](NC(=O)[C@@H]5NC6(CCCCC6)[C@@]6(C(=O)Nc7cc(Cl)ccc76)[C@H]5c5cccc(Cl)c5F)CC4)CC3)cc21]

In some embodiments, Compound A or a pharmaceutically acceptable salt thereof, is in amorphous form. In some embodiments, Compound A or a pharmaceutically acceptable salt thereof, is in crystalline form.

As used herein, the term “FLT3 inhibitor” includes, but is not limited to compounds having inhibitory activity against FMS-like Tyrosine Kinase 3 protein (FLT3), including but not limited to sunitinib, lestaurtinib, tandutinib, crenolanib, gilteritinib, midostaurin, quizartinib, and sorafenib, FLX925, and G-749.

As used herein, the term “inhibitor” is defined as a compound that binds to and/or inhibits MDM2 protein with measurable affinity. In certain embodiments, an inhibitor has an IC₅₀ and/or binding constant of less than about 50 μM, less than about 1 μM, less than about 500 nM, less than about 100 nM, less than about 10 nM, or less than about 1 nM.

As used herein, the term “MDM2 degrader” refers to an agent that degrades MDM2 protein. Various MDM2 degraders have been described previously, for example, in WO 2021/188948, the contents of which are incorporated herein by reference in their entireties. In certain embodiments, an MDM2 degrader has an DC50 of less than about 50 μM, less than about 1 μM, less than about 500 nM, less than about 100 nM, less than about 10 nM, or less than about 1 nM. In certain embodiments, the MDM2 degrader is Compound A disclosed herein.

The term “patient,” as used herein, means an animal, preferably a mammal, and most preferably a human.

As used herein, the term “MEK inhibitor” includes, but is not limited to compounds having inhibitory activity against mitogen-activated protein kinase kinase enzymes MEK1 and/or MEK2, including but not limited to binimetinib, cobimetinib, selumetinib, trametinib, mirametinib (PD-325901), and TAK-733.

As used herein, the term “mg/kg” or “mpk” refers to the milligram of medication (for example, Compound A) per kilogram of the body weight of the subject taking the medication.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

The term “pharmaceutically acceptable excipient or carrier” refers to a non-toxic excipient or carrier that does not destroy the pharmacological activity of the compound with which it is formulated. Pharmaceutically acceptable excipient or carrier that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The term “therapeutically effective amount” or “therapeutically effective dosage” as used herein refers to an amount of Compound A that is sufficient to treat the stated disease, disorder, or condition or have the desired stated effect on the disease, disorder, or condition or one or more mechanisms underlying the disease, disorder, or condition in a subject. In certain embodiments, when Compound A is administered for the treatment of a solid cancer or hematological malignancy, therapeutically effective amount refers an amount of Compound A which, upon administration to a subject, treats or ameliorates the solid cancer or hematological malignancy in the subject, or exhibits a detectable therapeutic effect in the subject that results in partial to complete tumor regression.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

The phase “woman of childbearing potential” (WOCBP) are considered fertile: 1. Following menarche; 2. From the time of menarche until becoming postmenopausal unless permanently sterile. A postmenopausal state is defined as no menses for 12 months without an alternative medical cause. A high follicle-stimulating hormone (FSH) level in the postmenopausal range may be used to confirm a postmenopausal state in women not using hormonal contraception or hormonal replacement therapy (HRT). However, in the absence of 12 months of amenorrhea, confirmation with more than one FSH measurement is required. Females on HRT and whose menopausal status is in doubt will be required to use one of the non-estrogen hormonal highly effective contraception methods if they wish to continue their HRT during the study. Otherwise, they must discontinue HRT to allow confirmation of postmenopausal status before study enrollment. Permanent sterilization methods (for the purpose of this study) include: documented hysterectomy; documented bilateral salpingectomy’ documented bilateral oophorectomy; for individuals with permanent infertility due to an alternate medical cause other than the above, (e.g., Mullerian agenesis, androgen insensitivity, gonadal dysgenesis), Investigator discretion should be applied to determining study entry.

### 3. Description of Exemplary Embodiments

The present disclosure provides a method for treating a relapsed and/or refractory solid cancer or hematological malignancy. In some embodiments, the present disclosure provides a method for treating a relapsed and/or refractory acute lymphoblastic leukemia (ALL) or acute myeloid leukemia (AML) in a patient, comprising administering to the patient a therapeutically effective amount of Compound A, or a pharmaceutically acceptable salt thereof, or a liquid formulation thereof as described herein.

In some embodiments, the present disclosure provides a liquid formulation, which comprises Compound A, or a pharmaceutically acceptable salt thereof, and a pharmaceutically acceptable excipient and/or carrier. In some embodiments, the present disclosure provides a unit dosage form, which comprises Compound A, or a pharmaceutically acceptable salt thereof, and a pharmaceutically acceptable excipient and/or carrier.

In some embodiments, a patient has a solid tumor (i.e., a solid tumor patient). In some embodiments, a solid tumor patient has a relapsed and/or refractory solid tumor.

In some embodiments, a solid tumor patient is male or female aged ≥18 years.

In some embodiments, a solid tumor patient has histologically or pathologically confirmed solid tumor.

In some embodiments, a method for treating a solid tumor patient, such as a relapsed and/or refractory solid tumor patient, of the invention comprises analyzing fresh or archival formalin fixed paraffin embedded (FFPE) tumor tissue or 15 slides preferably collected within ideally 6 months prior to first dose of compound A as described herein. In some embodiments, when archival tissue/slides/blocks are not available, the method for treating a solid tumor patient, such as a relapsed and/or refractory solid tumor patient, of the invention comprises preforming a pre-dose biopsy.

In some embodiments, a solid tumor patient has relapsed and/or refractory disease to at least two prior standard of care treatment or indications for whom standard therapies are not available.

In some embodiments, a solid tumor patient has at least one bi-dimensionally measurable disease site. In some embodiments, a bi-dimensionally measurable disease site, or a lesion, must have a greatest transverse diameter of at least 1.5 cm and greatest perpendicular diameter of at least 1.0 cm at baseline.

In some embodiments, a solid tumor patient has Eastern Cooperative Oncology Group (ECOG) performance status 0-2.

In some embodiments, a solid tumor patient has adequate organ function as defined by one or more of the following:
a) absolute neutrophil count (ANC) ≥1000/μL;
b) hemoglobin ≥8 g/dL (for those patients undergoing red blood cell [RBC]transfusion, hemoglobin must be evaluated after at least 14 days after the last RBC transfusion);
c) platelet count ≥100,000/μL (assessed ≥7 days following last platelet transfusion in patients with thrombocytopenia requiring platelets);
d) aspartate aminotransferase (AST), alanine transaminase (ALT) ≤3× upper limit of normal (ULN) or <5×ULN in cases of documented liver metastases;
e) total serum bilirubin ≤2.5×ULN or <5×ULN if secondary to Gilbert's syndrome or documented liver metastases; and
f) serum creatinine clearance ≥60 mL/min either measured or calculated using standard Cockcroft-Gault formula).

In some embodiments, a solid tumor patient is a women of child-bearing potential (WOCBP) and uses highly effective contraceptive methods for the duration of the treatment with Compound A as described herein and 6 months after the last dose of the treatment with Compound A as described herein.

In some embodiments, the WOCBP solid tumor patient uses a negative serum pregnancy test at screening and a negative serum or urine pregnancy test within 72 hours prior to first dose of Compound A as described herein.

In some embodiments, a solid tumor patient is a male and uses highly effective contraceptive methods for the duration of the treatment with Compound A as described herein and for 6 months after the last dose of Compound A as described herein if the partner is a WOCBP.

In some embodiments, a solid tumor patient has no history or suspicion of central nervous system (CNS) metastases.

In some embodiments, a solid tumor patient has no history of or active concurrent malignancy unless the patient has been disease-free for ≥2 years. Exceptions to the ≥2-year time limit include treated basal cell or localized squamous cell skin carcinoma, localized prostate cancer, or other localized carcinomas such as carcinoma in situ of cervix, breast, or bladder. In some embodiments, a solid tumor patient has history of or active concurrent malignancy selected from basal cell or localized squamous cell skin carcinoma, localized prostate cancer, and other localized carcinomas such as carcinoma in situ of cervix, breast, or bladder, and has not been disease-free for ≥2 years.

In some embodiments, a solid tumor patient has recovered from any clinically significant AEs of previous treatments to pre-treatment baseline or Grade 1 prior to first dose of Compound A as described herein.

In some embodiments, a solid tumor patient has no ongoing unstable cardiovascular function as defined by one or more of the following:
a) symptomatic ischemia;
b) uncontrolled clinically significant conduction abnormalities (i.e., ventricular tachycardia on anti-arrhythmic drugs is excluded; 1l degree atrioventricular block or asymptomatic left anterior fascicular block/right bundle branch block will not be excluded);
c) congestive heart failure of New York Heart Association Class ≥III;
d) myocardial infarction within 3 months prior to Screening; and
e) congenital long QT syndrome, or a QT interval corrected by Fridericia's formula (QTcF) ≥450 ms (average of triplicate electrocardiograms) at Screening and/or on C1D1 (pre-dose) with the exception of a documented bundle branch block or unless secondary to pacemaker.

In some embodiments, a solid tumor patient has no history of thromboembolic or cerebrovascular event (i.e., transient ischemic attacks, cerebrovascular accidents, pulmonary emboli, or clinically significant deep vein thrombosis) within 1 year prior to treatment with Compound A described herein.

In some embodiments, a solid tumor patient has no active severe infection that required anti-infective therapy during screening visits or on their first day of administration of Compound A described herein. In some embodiments, a solid tumor patient does not have a fever >38.5° C. during screening visits or on their first day of administration of Compound A described herein. In some embodiments, a solid tumor patient has a tumor fever during screening visits or on their first day of administration of Compound A described herein.

In some embodiments, a solid tumor patient has no history positive hepatitis B and/or hepatitis C serology or known seropositivity for human immunodeficiency virus (HIV).

In some embodiments, a solid tumor patient has no positive severe acute respiratory syndrome coronavirus-2 (SARS-CoV-2) test prior to treatment with Compound A described herein.

In some embodiments, a solid tumor patient has no concurrent medical conditions including psychiatric disorders.

In some embodiments, a solid tumor patient is not pregnant or breast feeding.

In some embodiments, a solid tumor patient is not receiving radiation treatment within 4 weeks prior to first dose of Compound A described herein.

In some embodiments, a solid tumor patient has not received major surgery requiring general anesthesia within 4 weeks prior to first dose of Compound A described herein.

In some embodiments, a solid tumor patient has not received live vaccine within 1 month prior to the first dose of Compound A described herein.

In some embodiments, a solid tumor patient has not had exposure to investigational or non-investigational anti-cancer therapy within 4 weeks or within at least 5 half-lives (up to a maximum of 4 weeks) prior to the first dose of Compound A described herein, whichever is longer.

In some embodiments, a solid tumor patient has had exposure to low dose steroids (oral prednisone or equivalent <20 mg/day), localized non-CNS radiotherapy, previous hormonal therapy with luteinizing hormone-releasing hormone (LHRH) agonists for prostate cancer, and/or treatment with bisphosphonates and RANKL inhibitors within 4 weeks or within at least 5 half-lives (up to a maximum of 4 weeks) prior to the first dose of Compound A described herein, whichever is longer.

In some embodiments, a solid tumor patient has not received any SARS-CoV-2 vaccine within 14 days prior to first dose of Compound A described herein. In some embodiments, a solid tumor patient has completed a course of SARS-CoV-2 vaccine 14 days or more prior to first dose of Compound A described herein.

In some embodiments, the patient is diagnosed with AML based on WHO classification that is Relapsed or Refractory to standard therapy or for which no standard therapies are anticipated to result in a durable remission or patients who are unsuitable to receive standard therapy due to age, performance status (PS), and/or adverse risk factors. In some embodiments, a patient has AML (i.e., an AML patient). In some embodiments, an AML patient has relapsed and/or refractory AML.

In some embodiments, the patient is diagnosed with ALL based on/classification patients with Relapsed or Refractory disease who are due to receive either salvage 1 or salvage 2 therapy. In some embodiments, Ph+ ALL patients have failed at least 1 second generation tyrosine kinase inhibitor. In some embodiments, a patient has ALL (i.e., an ALL patient). In some embodiments, an ALL patient has relapsed and/or refractory ALL.

In some embodiments, an AML or ALL patient is diagnosed with relapsed/progressed high/very high risk_MDS (score ≥4.5) according to IPSS-R risk stratification, or is intolerant to established therapy known to provide clinical benefit for their condition (i.e., subjects must not be candidates for regimens known to provide clinical benefit).

In some embodiments, the ALL patients comprise bone marrow involvement ≥5% lymphoblasts.

In some embodiments, the relapsed and/or refractory AML patients are defined as one or more of the following:
a) patients who relapsed after transplantation;
b) patients in second or later relapse;
c) patients who are refractory to initial induction or reinduction treatment; and
d) patients who relapsed within 1 year of initial treatment.

In some embodiments, the AML patients are defined as one or more of the following:
a) hydroxyurea allowed prior to Day 1 to keep peripheral blast count ≤10,000/μL; but must be discontinued 24 hours prior to first dose of Compound A described herein.
b) patients must have been off previous anti leukemia therapy for at least 2 weeks or 5 half-lives, whichever is shorter if the immediate prior regimen included only weekly chemotherapy; or 4 weeks from any therapy with therapeutic biologics and from any type of investigational therapy prior to the first dose of Compound A described herein.

In some embodiments, an AML or ALL patient has a life expectancy ≥12 weeks.

In some embodiments, an AML or ALL patient is ≥18 years old.

In some embodiments, an AML or ALL patient has ECOG performance status: 0-2.

In some embodiments, an AML or ALL patient has resolved acute effects of any prior therapy to baseline severity or Grade ≤1 CTCAE except for AEs not constituting a safety risk.

In some embodiments, an AML or ALL patient has received radiotherapy at least 4 weeks prior to the first dose of Compound A as described herein. In some embodiments, an AML or ALL patient has passed nadir white blood cell (WBC) and platelet counts, has full recovery or stabilization of absolute neutrophil counts (ANC) and platelet counts, and recovery of ANC counts from prior toxicity.

In some embodiments, an AML or ALL patient has adequate organ function at Screening defined by one or more of the following:
a) aspartate aminotransferase (AST), alanine transaminase (ALT)≤3× upper limit of normal (ULN), unless considered due to leukemic disease;
b) total serum bilirubin ≤1.5×ULN unless considered secondary to Gilbert's syndrome or leukemic disease;
c) serum creatinine clearance ≥50 mL/min either measured or calculated using standard Cockcroft-Gault formula);
d) platelet count ≥20,000/uL (transfusions to achieve this level are allowed).

In some embodiments, an AML or ALL patient has a baseline platelet count of <20,000/uL due to underlying malignancy.

In some embodiments, an AML or ALL patient is a women of child-bearing potential (WOCBP) and uses highly effective contraceptive methods for the duration of treatment with Compound A described herein and 6 months after the last dose of Compound A described herein.

In some embodiments, the WOCBP AML or ALL patient has a negative serum pregnancy test at screening and a negative serum or urine pregnancy test within 72 hours prior to first dose of Compound A described herein.

In some embodiments, an AML or ALL patient is a male using highly effective contraceptive methods during treatment with Compound A described herein and for 6 months after the last dose of Compound A described herein treatment if the partner is a WOCBP.

In some embodiments, an AML or ALL patient has documented TP53^{WT} disease.

In some embodiments, an AML patient does not have a diagnosis of acute promyelocytic leukemia (APL). In some embodiments, an AML patient does not have AML with known central nervous system (CNS) involvement, unless the patient has completed treatment for the CNS disease, has recovered from the acute effects of therapy prior to study entry, and is neurologically stable.

In some embodiments, an ALL patient does not have isolated extramedullary relapse.

In some embodiments, an ALL patient does not have Burkitt's or mixed lineage leukemia.

In some embodiments, an AML or ALL patient does not have active CNS leukemia. In some embodiments, a method for treating AML or ALL of the invention comprises performing a lumbar puncture to an AML or ALL patient with symptoms of CNS disease to rule out CNS disease.

In some embodiments, an AML or ALL patient does not have prior chemotherapy within ≤2 weeks of first dose of Compound A as described herein.

In some embodiments, an AML or ALL patient has received steroids, hydroxyurea, oral mercaptopurine, methotrexate, vincristine, thioguanine, and/or tyrosine kinase inhibitors within 2 weeks of the first dose of Compound A described herein as maintenance or to reduce the peripheral blast count.

In some embodiments, an AML or ALL patient does not have prior craniospinal radiation. In some embodiments, an AML or ALL patient has received other concurrent therapy for CNS prophylaxis or treatment of CNS relapse.

In some embodiments, an AML or ALL patient does not have peripheral lymphoblasts >10,000/μL. In some embodiments, an AML or ALL patient has received a treatment with hydroxurea and/or steroids within 2 weeks of first dose of Compound A described herein to reduce the WBC count.

In some embodiments, an AML or ALL patient does not have known systemic vasculitides (e.g., Wegener's granulomatosis, polyarteritis nodosa, systemic lupus erythematosus), primary or secondary immunodeficiency (such as HIV infection or severe inflammatory disease).

In some embodiments, an AML or ALL patient does not have chronic graft versus host disease (GVHD), active GVHD with other than Grade 1 skin involvement, or GVHD requiring immunosuppressive treatment. In some embodiments, an AML or ALL patient has GVHD and has received immunosuppressive treatment at least 2 weeks prior to the first dose of Compound A described herein.

In some embodiments, an AML or ALL patient is not known to be refractory to platelet or packed red cell transfusions per institutional guidelines.

In some embodiments, an AML or ALL patient is not within 3 months post allogenic hematopoietic stem cell transplant or within 30 days post autologous stem cell transplant prior to the first dose of Compound A described herein. In some embodiments, an AML or ALL patient has recovered from transplant-associated toxicities prior to the first dose of Compound A described herein.

In some embodiments, an AML or ALL patient does not have an active severe infection that required anti-infective therapy or with a fever >38.5° C. during screening visits or on their first day of administration of Compound A described herein. In some embodiments, an AML or ALL patient has a tumor fever during screening visits or on their first day of administration of Compound A described herein.

In some embodiments, an AML or ALL patient does not have evidence of uncontrolled current serious infection (including sepsis, bacteremia, fungemia, or history (within 4 months) of deep tissue infections such as fasciitis or osteomyelitis.

In some embodiments, an AML or ALL patient does not have current or chronic hepatitis B or C infection as evidenced by hepatitis B surface antigen and anti-hepatitis C antibody positivity, respectively, or known seropositivity for human immunodeficiency virus (HIV).

In some embodiments, an AML or ALL patient has not participated in other studies involving investigational drug(s) within 4 weeks prior to the first dose of Compound A described herein.

In some embodiments, an AML or ALL patient has not had major surgery within 4 weeks of study entry.

In some embodiments, an AML or ALL patient is not receiving chronic systemic corticosteroid treatment. In some embodiments, an AML or ALL patient receives topical applications, inhaled sprays, eye drops, local injections of corticosteroids and systemic steroids required for acute medical interventions.

In some embodiments, an AML or ALL patient does not have other severe acute or chronic medical or psychiatric condition, including recent (within the past year) or active suicidal ideation or behavior, or laboratory abnormality.

In some embodiments, an AML or ALL patient has not had any of the following in the previous 6 months: myocardial infarction, congenital long QT syndrome, Torsades de pointes, arrhythmias (including sustained ventricular tachyarrhythmia and ventricular fibrillation), right bundle branch block and left anterior hemiblock (bifascicular block), unstable angina, coronary/peripheral artery bypass graft, symptomatic congestive heart failure (CHF New York Heart Association class III or IV), cerebrovascular accident, transient ischemic attack, or symptomatic pulmonary embolism.

In some embodiments, an AML or ALL patient does not have ongoing cardiac dysrhythmias of NCI CTCAE Grade 32 or QTcF interval >470 msec (based on average of 3 consecutive ECGs) at screening.

In some embodiments, an AML or ALL patient does not have left ventricular ejection fraction (LVEF)<40% by echocardiogram (ECHO) or multi gated acquisition (MUGA) scan.

In some embodiments, an AML or ALL patient docs not have history of chronic liver disease or suspected alcohol abuse.

In some embodiments, an AML or ALL patient does not have history of hepatic veno-occlusive disease (VOD) or sinusoidal obstruction syndrome (SOS).

In some embodiments, a patient with relapsed and/or refractory (R/R) high grade myeloid malignancies, acute lymphocytic leukemia (ALL), R/R lymphoma, or R/R solid tumors has an Eastern Cooperative Oncology Group (ECOG) performance status of 0-2.

In some embodiments, a patient with relapsed and/or refractory (R/R) high grade myeloid malignancies, acute lymphocytic leukemia (ALL), R/R lymphoma, or R/R solid tumors has resolved acute effects of any prior therapy to baseline severity or Grade ≤1 NCI CTCAE.

In some embodiments, a patient with relapsed and/or refractory (R/R) high grade myeloid malignancies, acute lymphocytic leukemia (ALL), R/R lymphoma, or R/R solid tumors has adequate organ and bone marrow function in the absence of growth factors.

In some embodiments, a patient with R/R lymphoma or R/R solid tumors has histologically or pathologically confirmed solid tumor or lymphoma.

In some embodiments, a patient with R/R lymphoma or R/R solid tumors has relapsed and/or refractory disease to at least two prior standard-of-care treatments or tumors for whom standard therapies are not available.

In some embodiments, a patient with R/R lymphoma or R/R solid tumors has advanced high grade myeloid malignancies, and Acute Lymphocytic Leukemia.

In some embodiments, a patient with R/R lymphoma or R/R solid tumors has primary diagnosis of AML, ALL, Relapsed/progressed high-risk Myelodysplastic Syndromes (MDS), Myelodysplastic/myeloproliferative neoplasms (MDS/MPN).

In some embodiments, a patient with R/R lymphoma or R/R solid tumors has at least 4 weeks since radiotherapy prior to the first dose of Compound A described herein.

In some embodiments, a patient with relapsed and/or refractory (R/R) high grade myeloid malignancies, acute lymphocytic leukemia (ALL), R/R lymphoma, or R/R solid tumors does not have ongoing unstable cardiovascular function.

In some embodiments, a patient with relapsed and/or refractory (R/R) high grade myeloid malignancies, acute lymphocytic leukemia (ALL), R/R lymphoma, or R/R solid tumors has not had major surgery within 4 weeks of study entry.

In some embodiments, a patient with relapsed and/or refractory (R/R) high grade myeloid malignancies, acute lymphocytic leukemia (ALL), R/R lymphoma, or R/R solid tumors does not have history of or active concurrent malignancy unless disease-free for ≥2 years.

In some embodiments, a patient with relapsed and/or refractory (R/R) high grade myeloid malignancies, acute lymphocytic leukemia (ALL), R/R lymphoma, or R/R solid tumors has not had exposures to anticancer therapy within 2 weeks or 5 half-lives whichever is shorter; or 4 weeks from any biologics/immunotherapies or any investigational therapy prior to the first dose of Compound A described herein.

In some embodiments, a patient with R/R lymphoma or R/R solid tumors has no known active uncontrolled or symptomatic central nervous system (CNS) metastases.

In some embodiments, a patient with R/R lymphoma or R/R solid tumors has no known autologous hematopoietic stem cell transplant (HSCT) within six months prior to first dose of study drug or participant has progressed within six months from the day of stem cell.

In some embodiments, a patient with R/R lymphoma or R/R solid tumors has no known prior allogeneic hematopoietic stem cell transplant.

In some embodiments, a patient with relapsed and/or refractory (R/R) high grade myeloid malignancies or acute lymphocytic leukemia (ALL) does not have active CNS leukemia.

In some embodiments, a patient with relapsed and/or refractory (R/R) high grade myeloid malignancies or acute lymphocytic leukemia (ALL) has no known prior chemotherapy/radiation within ≤2 weeks of first dose of study drug.

In some embodiments, a patient with relapsed and/or refractory (R/R) high grade myeloid malignancies or acute lymphocytic leukemia (ALL) has no known systemic vasculitides (e.g., Wegener's granulomatosis, polyarteritis nodosa, or systemic lupus erythematosus).

In some embodiments, a patient with relapsed and/or refractory (R/R) high grade myeloid malignancies or acute lymphocytic leukemia (ALL) is not within 3 months post allogenic hematopoietic stem cell transplant or within 30 days post autologous stem cell transplant, and the patient has not recovered from transplant-associated toxicities.

In some embodiments, a patient with relapsed and/or refractory (R/R) high grade myeloid malignancies or acute lymphocytic leukemia (ALL) does not have active or chronic graft versus host disease (GVHD) or on treatment for GVHD.

In some embodiments, a method of the present invention comprises intravenously administering a liquid formulation as described herein. In some embodiments, a method of the present invention comprises administering a unit dosage form as described herein. In some embodiments, a method of the present invention comprises administering daily to a patient a liquid formulation or a unit dosage form as described herein.

### Liquid Formulations

According to one embodiment, the invention provides a liquid formulation or unit dosage form comprising Compound A, or a pharmaceutically acceptable derivative thereof and a pharmaceutically acceptable excipient (e.g., a buffer) and/or carrier (e.g., water). The amount of Compound A in liquid formulations or unit dosage forms of this invention is such that it is effective to measurably degrade and/or inhibit MDM2 protein, or a mutant thereof, in a patient. In certain embodiments, a liquid formulation or unit dosage form of this invention is formulated for administration to a patient in need of such composition. In some embodiments, liquid formulation or unit dosage form of this invention is formulated for parenteral (e.g., intravenous) administration to a patient.

The liquid formulation or unit dosage form of the present invention may be administered parenterally by injection, infusion or implantation (intravenous, intramuscular, subcutaneous, or the like) as the liquid formulation or in unit dosage forms or via suitable delivery devices or implants containing conventional, non-toxic pharmaceutically acceptable carriers and adjuvants. In certain embodiments, the the liquid formulation or unit dosage form of the present invention is administered by intravenous transfusion.

In some embodiments, a provided liquid formulation for parenteral use are provided in unit dosage forms (e.g., in single-dose ampoules), or in vials containing several doses and in which a suitable preservative may be added. Typically, such compositions can be prepared as injectable formulations, for example, solutions or suspensions; solid and liquid forms suitable for using to prepare solutions or suspensions upon the addition of a reconstitution or dilution medium prior to injection; emulsions, such as water-in-oil (w/o) emulsions, oil-in-water (o/w) emulsions, and microemulsions thereof, liposomes, or emulsomes. In some embodiments, the liquid formulation or unit dosage forms thereof are administered intravenously.

In some embodiments, the liquid formulations or unit dosage form comprising Compound A of present invention or pharmacologically acceptable salts thereof can be prepared in water or another solvent or dispersing medium suitably mixed with one or more pharmaceutically acceptable excipients including, but not limited to buffers, surfactants, solubilizing agents, dispersants, emulsifiers, viscosity modifying agents, and combination thereof.

In some embodiments, a liquid formulation or unit dosage form of the invention comprises Compound A, or a pharmaceutically acceptable salt thereof, at a concentration of about 0.05-5% w/w of the total weight of the formulation or unit dosage form. In some embodiments, a liquid formulation or unit dosage form of the invention comprises Compound A, or a pharmaceutically acceptable salt thereof, at a concentration of about 0.05-0.5%, about 0.1-1.0%, about 0.6-1.4%, about 0.7-1.3%, about 0.8-1.2%, or about 0.9-1.1% w/w of the total weight of the formulation or unit dosage form. In some embodiments, a liquid formulation or unit dosage form of the invention comprises Compound A, or a pharmaceutically acceptable salt thereof, at a concentration of about 0.60%, about 0.65%, about 0.70%, about 0.75%, about 0.80%, about 0.85%, about 0.90%, about 0.95%, about 1.00%, about 1.05%, about 1.10%, about 1.15%, about 1.20%, about 1.25%, about 1.30%, about 1.35%, about 1.40%, about 1.45%, or about 1.50% w/w of the total weight of the formulation or unit dosage form.

In some embodiments, a liquid formulation or unit dosage form of the invention comprises Compound A, or a pharmaceutically acceptable salt thereof, at a concentration of about 1-20 mg/mL. In some embodiments, a liquid formulation or unit dosage form of the invention comprises Compound A, or a pharmaceutically acceptable salt thereof, at a concentration of about 1-5 mg/mL, about 1-10 mg/mL, about 6-14 mg/mL, about 6.5-13.5 mg/mL, about 7-13 mg/mL, about 7.5-12.5 mg/mL, about 8-12 mg/mL, about 8.5-11.5 mg/mL, about 9-11 mg/mL, or about 9.5-10.5 mg/mL. In some embodiments, a liquid formulation or unit dosage form of the invention comprises Compound A, or a pharmaceutically acceptable salt thereof, at a concentration of about 8 mg/mL, about 8.5 mg/mL, about 9 mg/mL, about 9.5 mg/mL, about 10 mg/mL, about 10.5 mg/mL, about 11 mg/mL, about 11.5 mg/mL, or about 12 mg/mL.

In some embodiments, the liquid formulations or unit dose forms are packaged in solutions with one or more aqueous buffer. In some embodiments, the liquid formulations or unit dosage forms are packaged in solutions with sterile isotonic aqueous buffers. In some embodiments, the liquid formulations or unit dosage forms are buffered at about pH 5-8 for parenteral administration upon dilution. Suitable buffers or buffering agents include, but are not limited to, phosphate buffers, citrate buffers, acetate buffers, histidine buffers, or succinate buffers. In some embodiments, the buffer is one or more phosphate buffer.

In some embodiments, the liquid formulation or unit dosage form may also include a solubilizing agent. The components of the formulation can be either separately or mixed together in unit dosage form, for example, as a dry lyophilized powder (which can be reconstituted before use with a carrier such as saline) or concentrated solution in a hermetically sealed container such as an ampoule or sachet indicating the amount of active agent. If the composition is to be administered by infusion, it can be dispensed with an infusion bottle or bag containing sterile pharmaceutical grade water or saline. Where the formulation is administered by injection, an ampoule of sterile water or saline can be provided so that the ingredients may be mixed prior to injection.

In some embodiments, the carrier can be a solvent or dispersion medium containing, for example, water, ethanol, one or more polyols (e.g., glycerol, propylene glycol, and liquid polyethylene glycol), oils, such as vegetable oils (e.g., peanut oil, corn oil, sesame oil, etc.), and combinations thereof. The proper fluidity can be maintained, for example, by the use of a coating, such as lecithin, by the maintenance of the required particle size in the case of dispersion and/or by the use of surfactants. In many cases, it will be preferable to include isotonic agents, for example, sugars or sodium chloride. In some embodiments, the carrier is ethanol, a polyol, or a mixture of ethanol and a polyol. In some embodiments, the ethanol is 200 proof (i.e., food grade) ethanol. In some embodiments, the polyol is a liquid polyethylene glycol, such as polyethylene glycol 400.

In some embodiments, a liquid formulation or unit dosage form of the invention comprises one or more carriers (e.g., ethanol and a polyol) at a concentration of about 60-90% w/w of the total weight of the formulation or unit dosage form. In some embodiments, a liquid formulation or unit dosage form of the invention comprises one or more carriers (e.g., ethanol and a polyol) at a concentration of about 60-80/c, about 70-85%, or about 75-90% w/w of the total weight of the formulation or unit dosage form. In some embodiments, a liquid formulation or unit dosage form of the invention comprises one or more carriers (e.g., ethanol and a polyol) at a concentration of about 70%, about 71%, about 72%, about 73%, about 74%, about 75%, about 76%, about 77%, about 78%, about 79%, about 80% c, about 81%, about 82%, about 83%, about 84%, about 85%, about 86%, about 87%, about 88%, about 89%, or about 90% w/w of the total weight of the formulation or unit dosage form. In some embodiments, a liquid formulation or unit dosage form of the invention comprises at first carrier (e.g., a polyol) at a concentration of about 60-80% of the total weight of the formulation or unit dosage form. In some embodiments, a liquid formulation or unit dosage form of the invention comprises a second carrier (e.g, ethanol) at a concentration of about 5-20% of the total weight of the formulation or unit dosage form. In some embodiment, a liquid formulation or unit dosage form of the invention comprises a first carrier and a second carrier in a ratio of about 1:10 to about 10:1. In some embodiments, the first carrier is a polyol (e.g., polyethylene glycol 400) and the second carrier is ethanol in a ratio of about 5:1 to about 9:1, for example about 6:1, about 7:1, or about 8:1.

In some embodiments, a liquid formulation or unit dosage form of the invention comprises a carrier (e.g., ethanol and a polyol) at a concentration of about 400-1500 mg/mL. In some embodiments, a liquid formulation or unit dosage form of the invention comprises Compound A, or a pharmaceutically acceptable salt thereof at a concentration of about 400-600 mg/mL, about 500-700 mg/mL, about 600-800 mg/mL, about 700-900 mg/mL, about 800-1000 mg/mL, about 900-1100 mg/mL, about 1000-1200 mg/mL, about 1100-1300 mg/mL, about 1200-1400 mg/mL, or about 1300-1500 mg/mL. In some embodiments, a liquid formulation or unit dosage form of the invention comprises a first carrier (e.g., a polyol) at a concentration of about 650-850 mg/mL. In some embodiments, a liquid formulation or unit dosage form of the invention comprises a second carrier (e.g., ethanol) at a concentration of about 50-150 mg/mL.

Suitable surfactants may be anionic, cationic, amphoteric or nonionic surface-active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene, and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates (e.g. polysorbate 80), polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-beta-alanine, sodium N-laurylβiminodipropionate, myristoamphoacetate, lauryl betaine, and lauryl sulfobetaine.

In some embodiments, a liquid formulation or unit dosage form of the invention comprises a surfactant (e.g., polysorbate 80) at a concentration of about 10-30% w/w of the total weight of the formulation or unit dosage form. In some embodiments, a liquid formulation or unit dosage form of the invention comprises a surfactant (e.g., polysorbate 80) at a concentration of about 10-20%, about 15-25%, or about 20-30% w/w of the total weight of the formulation or unit dosage form. In some embodiments, a liquid formulation or unit dosage form of the invention comprises a surfactant (e.g., polysorbate 80) at a concentration of about 15%, about 16%, about 17%, about 18%, about 19%, about 20%, about 21%, about 22%, about 23%, about 24%, about 25%, about 26%, about 27%, about 28%, about 29%, or about 30% w/w of the total weight of the formulation or unit dosage form.

In some embodiments, a liquid formulation or unit dosage form of the invention comprises a surfactant (e.g., polysorbate 80) at a concentration of about 100-500 mg/mL of the total weight of the formulation or unit dosage form. In some embodiments, a liquid formulation or unit dosage form of the invention comprises a surfactant (e.g., polysorbate 80) at a concentration of about 100-300, about 200-400, or about 300-500 mg/mL. In some embodiments, a liquid formulation or unit dosage form of the invention comprises a surfactant (e.g., polysorbate 80) at a concentration of about 110, about 120, about 130, about 140, about 150, about 160, about 170, about 180, about 190, about 200, about 210, about 220, about 230, about 240, about 250, about 260, about 270, about 280, about 290, about 300, about 310, about 320, about 330, about 340, about 350, about 360, about 370, about 380, about 390, or about 400 mg/mL.

The formulation can contain a preservative to prevent the growth of microorganisms. Suitable preservatives include, but are not limited to, parabens, chlorobutanol, phenol, sorbic acid, and thimerosal. The formulation may also contain an antioxidant to prevent degradation of the active agent(s).

In some embodiments, a liquid formulation or unit dosage form of the invention comprises an antioxidant (e.g., butylated hydroxytoluene) at a concentration of about 0.001-0.1% w/w of the total weight of the formulation or unit dosage form. In some embodiments, a liquid formulation or unit dosage form of the invention comprises an antioxidant (e.g., butylated hydroxytoluene) at a concentration of about 0.001-0.01%, about 0.005-0.05%, or about 0.01-0.1% w/w of the total weight of the formulation or unit dosage form. In some embodiments, a liquid formulation or unit dosage form of the invention comprises an antioxidant (e.g., butylated hydroxytoluene) at a concentration of about 0.01-1 mg/mL. In some embodiments, a liquid formulation or unit dosage form of the invention comprises an antioxidant (e.g., butylated hydroxytoluene) at a concentration of about 0.01-0.1 mg/mL, about 0.05-0.5 mg/mL, or about 0.1-1 mg/mL.

In some embodiments, the present invention provides a unit dosage form, which is a liquid formulation of the present invention, as described above, with a volume of from about 1 mL to about 10 mL. In some embodiments, the present invention provides a unit dosage form, which is a liquid formulation of the present invention, as described above, with a volume of about 1 mL, about 2 mL, about 3 mL, about 4 mL, about 5 mL, about 6 mL, about 7 mL, about 8 mL, about 9 mL, or about 10 mL.

In some embodiments, a liquid formulation or unit dosage form of the invention comprises Compound A or a pharmaceutically acceptable salt thereof at about 0.5-1.5% w/w (e.g., about 0.9% w/w of Compound A), about 15-25% w/w surfactant (e.g., about 10% w/w polysorbate 80), about 60-80% w/w a first carrier (e.g., about 69% w/w polyethylene glycol 400), about 5-15% w/w a second carrier (e.g., about 10% w/w ethanol), and about 0.005-0.05% w/w antioxidant (e.g., about 0.01% w/w butylated hydroxytoluene) of the total weight of the formulation or unit dosage form. In some embodiments, a liquid formulation or unit dosage form of the invention is as depicted in Table 3, below.

Water-soluble polymers are often used in formulations for parenteral administration. Suitable water-soluble polymers include, but are not limited to, polyvinylpyrrolidone, dextran, carboxymethylcellulose, and polyethylene glycol.

In some embodiments, the liquid formulation may include a solubilizing agent. In some embodiments, the solubilizing agent is a cyclodextrin. Cyclodextrines include members of a family of cyclic oligosaccharides, composed of 5 or more α-D-glucopyranoside units linked between positions 1 and 4, as known for amylose, a fragment of starch. In some embodiments, the cyclodextrin is an α-cyclodextrin, β-cyclodextrin, and/or γ-cyclodextrin.

Sterile injectable solutions can be prepared by incorporating the active compounds in the required amount in the appropriate solvent or dispersion medium with one or more of the excipients listed above, as required, followed by filtered sterilization. Generally, dispersions are prepared by incorporating the various sterilized active ingredients into a sterile vehicle which contains the basic dispersion medium and the required other ingredients from those listed above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. The powders can be prepared in such a manner that the particles are porous in nature, which can increase dissolution of the particles. Methods for making porous particles are well known in the art.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated.

### Administration and Dosage

As described herein, the liquid formulations or unit dosage form comprising Compound A of present invention are administered intravenously. In some embodiments, Compound A or a pharmaceutically acceptable salt thereof is administered by an IV injection. In some embodiments, Compound A or a pharmaceutically acceptable salt thereof is administered by an IV infusion.

In some embodiments, the methods and uses described herein, such as the method of or use in treating a solid cancer or hematological malignancy in a patient in need thereof, is achieved by administering (e.g., intravenously) a therapeutically effective amount of Compound A, such as up to 100 mg in a single or multiple dosage units. In some embodiments, the method can include administering (e.g., intravenously), in a single or multiple dosage units ranging from about 1 to about 100 mg/dosage form, such as about 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 60 mg, 70 mg, 80 mg, 90 mg, or about 100 mg. For example, a liquid formulation can include 5 mg, 10 mg, 15 mg, 20 mg, 25 mg, 30 mg, 35 mg, 40 mg, 45 mg, or 50 mg/dosage form of Compound A or a pharmaceutically acceptable salt thereof.

In some embodiments, Compound A or a pharmaceutically acceptable salt thereof is intravenously administered at a dose of up to 5 mg to the patient. In some embodiments, Compound A or a pharmaceutically acceptable salt thereof is intravenously administered at a dose of up to 10 mg to the patient. In some embodiments, Compound A or a pharmaceutically acceptable salt thereof is intravenously administered at a dose of up to 15 mg to the patient. In some embodiments, Compound A or a pharmaceutically acceptable salt thereof is intravenously administered at a dose of up to 20 mg to the patient. In some embodiments, Compound A or a pharmaceutically acceptable salt thereof is intravenously administered at a dose of up to 25 mg to the patient. In some embodiments, Compound A or a pharmaceutically acceptable salt thereof is intravenously administered at a dose of up to 30 mg to the patient. In some embodiments, Compound A or a pharmaceutically acceptable salt thereof is intravenously administered at a dose of up to 35 mg to the patient. In some embodiments, Compound A or a pharmaceutically acceptable salt thereof is intravenously administered at a dose of up to 40 mg to the patient. In some embodiments, Compound A or a pharmaceutically acceptable salt thereof is intravenously administered at a dose of up to 45 mg to the patient. In some embodiments, Compound A or a pharmaceutically acceptable salt thereof is intravenously administered at a dose of up to 50 mg to the patient. In some embodiments, Compound A or a pharmaceutically acceptable salt thereof is intravenously administered at a dose of from about 10 mg to about 40 mg to the patient. In some embodiments, Compound A or a pharmaceutically acceptable salt thereof is intravenously administered at a dose of from about 20 mg to about 50 mg to the patient, such as about 30 mg, 35 mg, or 40 mg. In some embodiments, Compound A or a pharmaceutically acceptable salt thereof is intravenously administered at a dose of about 35 mg to the patient.

In some embodiments, a liquid pharmaceutical composition is provided, wherein, the pharmaceutically composition comprises 5 mg to about 50 mg of Compound A, or a pharmaceutically acceptable salt thereof, and one or more pharmaceutically acceptable excipient or carrier. In some embodiments, a liquid pharmaceutical composition is provided, wherein, the pharmaceutically composition comprises 25 mg to about 45 mg of Compound A, or a pharmaceutically acceptable salt thereof, and one or more pharmaceutically acceptable excipient or carrier.

In some embodiments, Compound A, or a pharmaceutically acceptable salt thereof, or a pharmaceutical composition thereof, is administered intravenously to a patient at a dose of up to about 30 mg/m². In some embodiments, Compound A, or a pharmaceutically acceptable salt thereof, or a pharmaceutical composition thereof, is administered intravenously to a patient at a dose of up to about 25 mg/m², or up to about 20 mg/m², or up to about 15 mg/m². In some embodiments, Compound A, or a pharmaceutically acceptable salt thereof, or a pharmaceutical composition thereof, is administered intravenously to a patient at a dose of about 1 mg/m² to about 5 mg/m², or about 3 mg/m² to about 8 mg/m², or about 5 mg/m² to about 10 mg/m², or about 7 mg/m² to about 12 mg/m², or about 10 mg/m² to about 15 mg/m², or about 12 mg/m² to about 7 mg/m², or about 15 mg/m² to about 20 mg/m², or about 17 mg/m² to about 22 mg/m², or about 20 mg/m² to about 25 mg/m², or about 22 mg/m² to about 27 mg/m². In some embodiments, Compound A, or a pharmaceutically acceptable salt thereof, or a pharmaceutical composition thereof, is administered intravenously to a patient at a dose of about 30 mg/m², about 27 mg/m², about 20 mg/m², about 17 mg/m², about 15 mg/m², about 12 mg/m², about 10 mg/m², about 7 mg/m², about 5 mg/m², about 3 mg/m², or about 1 mg/m².

In some embodiments, Compound A, or a pharmaceutically acceptable salt thereof, or a pharmaceutical composition thereof, is administered intravenously to a patient at a dose of up to about 0.8 mg/kg. In some embodiments, Compound A, or a pharmaceutically acceptable salt thereof, or a pharmaceutical composition thereof, is administered intravenously to a patient at a dose of up to about 0.6 mg/kg, or up to about 0.3 mg/kg, or up to about 0.1 mg/kg. In some embodiments, Compound A, or a pharmaceutically acceptable salt thereof, or a pharmaceutical composition thereof, is administered intravenously to a patient at a dose of about 0.01 mg/kg to about 0.05 mg/kg, or about 0.03 mg/kg to about 0.08 mg/kg, or about 0.05 mg/kg to about 0.1 mg/kg, or about 0.07 mg/kg to about 0.12 mg/kg, or about 0.1 mg/kg to about 0.15 mg/kg, or about 0.12 mg/kg to about 0.17 mg/kg. In some embodiments, Compound A, or a pharmaceutically acceptable salt thereof, or a pharmaceutical composition thereof, is administered intravenously to a patient at a dose of about 1 mg/kg, about 0.8 mg/kg, about 0.5 mg/kg, about 0.3 mg/kg, about 0.1 mg/kg, about 0.08 mg/kg, or about 0.06 mg/kg.

### Dosing Schedule

As provided herein, Compound A or a pharmaceutically acceptable salt thereof, or a pharmaceutical composition thereof, is administered to a patient at a dosing schedule appropriate to give the desired tumor regression effect with minimum side effects. In some embodiments, Compound A or pharmaceutical composition thereof is administered to a patient once every 1, 2, 3, 4, 5, 6, 7, 14, or 21 days. In some embodiments, Compound A or pharmaceutical composition thereof is administered to a patient daily (QD). In some embodiments, Compound A or pharmaceutical composition thereof is administered to a patient biweekly (BW). In some embodiments, Compound A or pharmaceutical composition thereof is administered to a patient weekly (QW). In some embodiments, Compound A or pharmaceutical composition thereof is administered to a patient every two weeks (Q2W). In some embodiments, Compound A or pharmaceutical composition thereof is administered to a patient every three weeks (Q3W).

In some embodiments, an IV infusion of a pharmaceutical composition of the invention lasts about 5-30 minutes. In some embodiments, an IV infusion of a pharmaceutical composition of the invention lasts about 30-90 minutes. In some embodiments, an IV infusion of a pharmaceutical composition of the invention lasts about 5, 10, 15, 20, 25, 30, 35, 40, 45, 50, 55, 60, 65, 70, 75, 80, 85, or 90 minutes. In some embodiments, an IV infusion of a pharmaceutical composition of the invention lasts about 2, 2.5, 3, 3.5, or 4 hours.

In some embodiments, a pharmaceutical composition of the invention is administered intravenously weekly at a dose of from about 0.1 mg/m² to about 30 mg/m². In some embodiments, a pharmaceutical composition of the invention is administered intravenously weekly at a dose of from about 1 mg/m² to about 10 mg/m².

### Methods and Uses for Treating Disease

Compound A and compositions described herein are useful for the degradation and/or inhibition of MDM2 protein activity.

Thus, in certain embodiments, the present invention provides a method for treating a MDM2-mediated disorder comprising the step of administering to a patient in need thereof Compound A of the present invention, or pharmaceutically acceptable composition thereof.

As used herein, the terms “MDM2-mediated” disorders, diseases, and/or conditions as used herein means any disease or other deleterious condition in which MDM2 protein or a mutant thereof, are known to play a role. Accordingly, another embodiment of the present invention relates to treating or lessening the severity of one or more diseases in which MDM2 protein or a mutant thereof, are known to play a role.

In some embodiments, the present invention provides a method for treating one or more disorders, diseases, and/or conditions wherein the disorder, disease, or condition is a cancer, a neurodegenerative disorder, a viral disease, an autoimmune disease, an inflammatory disorder, a hereditary disorder, a hormone-related disease, a metabolic disorder, conditions associated with organ transplantation, immunodeficiency disorders, a destructive bone disorder, a proliferative disorder, an infectious disease, a condition associated with cell death, thrombin-induced platelet aggregation, liver disease, pathologic immune conditions involving T cell activation, a cardiovascular disorder, or a CNS disorder.

In some embodiments, the cancer is selected from adrenal cancer, acinic cell carcinoma, acoustic neuroma, acral lentiginous melanoma, acrospiroma, acute eosinophilic leukemia, acute erythroid leukemia, acute lymphoblastic leukemia, acute megakaryoblastic leukemia, acute monocytic leukemia, acute promyelocytic leukemia, adenocarcinoma, adenoid cystic carcinoma, adenoma, adenomatoid odontogenic tumor, adenosquamous carcinoma, adipose tissue neoplasm, adrenocortical carcinoma, adult T-cell leukemia/lymphoma, aggressive NK-cell leukemia, AIDS-related lymphoma, alveolar rhabdomyosarcoma, alveolar soft part sarcoma, ameloblastic fibroma, anaplastic large cell lymphoma, anaplastic thyroid cancer, angioimmunoblastic T-cell lymphoma, angiomyolipoma, angiosarcoma, astrocytoma, rhabdoid (e.g., atypical teratoid rhabdoid tumor), B-cell chronic lymphocytic leukemia, B-cell prolymphocytic leukemia, B-cell lymphoma, basal cell carcinoma, bile duct cancer, biliary tract cancer, bladder cancer, blastoma, bone cancer, myelofibrosis, Brenner tumor, Brown tumor, Burkitt's lymphoma, breast cancer, brain cancer, carcinoma, carcinoma in situ, carcinosarcoma, cartilage tumor, cementoma, myeloid sarcoma, chondroma, chordoma, choriocarcinoma, choroid plexus papilloma, clear-cell sarcoma of the kidney, craniopharyngioma, cutaneous T-cell lymphoma, cervical cancer, colorectal cancer, Degos disease, desmoplastic small round cell tumor, diffuse large B-cell lymphoma, dysembryoplastic neuroepithelial tumor, dysgerminoma, embryonal carcinoma, endocrine gland neoplasm, endodermal sinus tumor, enteropathy-associated T-cell lymphoma, esophageal cancer, fetus in fetu, fibroma, fibrosarcoma, follicular lymphoma, follicular thyroid cancer, ganglioneuroma, gastrointestinal cancer, germ cell tumor, gestational choriocarcinoma, giant cell fibroblastoma, giant cell tumor of the bone, glial tumor, glioblastoma multiforme, glioma, gliomatosis cerebri, glucagonoma, gonadoblastoma, granulosa cell tumor, gynandroblastoma, gallbladder cancer, gastric cancer, hairy cell leukemia, hemangioblastoma, head and neck cancer, hemangiopericytoma, hematological malignancy, hepatoblastoma, hepatosplenic T-cell lymphoma, Hodgkin's lymphoma, non-Hodgkin's lymphoma, invasive lobular carcinoma, intestinal cancer, kidney cancer, laryngeal cancer, lentigo maligna, lethal midline carcinoma, leukemia, leydig cell tumor, liposarcoma, lung cancer, lymphangioma, lymphangiosarcoma, lymphoepithelioma, lymphoma, acute lymphocytic leukemia, acute myelogeous leukemia, chronic lymphocytic leukemia, liver cancer, small cell lung cancer, non-small cell lung cancer, MALT lymphoma, malignant fibrous histiocytoma, malignant peripheral nerve sheath tumor, malignant triton tumor, mantle cell lymphoma, marginal zone B-cell lymphoma, mast cell leukemia, mediastinal germ cell tumor, medullary carcinoma of the breast, medullary thyroid cancer, medulloblastoma, melanoma, meningioma, merkel cell cancer, mesothelioma, metastatic urothelial carcinoma, mixed Mullerian tumor, mucinous tumor, multiple myeloma, muscle tissue neoplasm, mycosis fungoides, myxoid liposarcoma, myxoma, myxosarcoma, nasopharyngeal carcinoma, neurinoma, neuroblastoma, neurofibroma, neuroma, nodular melanoma, ocular cancer, oligoastrocytoma, oligodendroglioma, oncocytoma, optic nerve sheath meningioma, optic nerve tumor, oral cancer, osteosarcoma, ovarian cancer, Pancoast tumor, papillary thyroid cancer, paraganglioma, pinealoblastoma, pineocytoma, pituicytoma, pituitary adenoma, pituitary tumor, plasmacytoma polyembryoma, precursor T-lymphoblastic lymphoma, primary central nervous system lymphoma, primary effusion lymphoma, primary peritoneal cancer, prostate cancer, pancreatic cancer, pharyngeal cancer, pseudomyxoma periotonei, renal cell carcinoma, renal medullary carcinoma, retinoblastoma, rhabdomyoma, rhabdomyosarcoma, Richter's transformation, rectal cancer, sarcoma, Ewing sarcoma, Schwannomatosis, seminoma, Sertoli cell tumor, sex cord-gonadal stromal tumor, signet ring cell carcinoma, skin cancer, Merkel cell carcinoma, small blue round cell tumors, small cell carcinoma, soft tissue sarcoma, somatostatinoma, soot wart, spinal tumor, splenic marginal zone lymphoma, squamous cell carcinoma, synovial sarcoma, Sezary's disease, small intestine cancer, squamous carcinoma, stomach cancer, T-cell lymphoma, testicular cancer, thecoma thyroid cancer, transitional cell carcinoma, throat cancer, urachal cancer, urogenital cancer, urothelial carcinoma, uveal melanoma, uterine cancer, verrucous carcinoma, visual pathway ghoma, vulvar cancer, vaginal cancer, Waldenstrom's macroglobulinemia, Warthin's tumor, and Wilms' tumor.

MDM2 hyperactivity, due to amplification/overexpression or mutational inactivation of the ARF locus, inhibits the function of wild-type p53 and can lead to the development of a wide variety of cancers. In some embodiments, the MDM2 hyperactivity which can be treated according to the methods of this invention is a human cancer. In some embodiments, the human cancer which can be treated according to the methods of this invention is selected from a solid cancer or hematological malignancy. In some embodiments, the wild-type p53 cancer is mesothelioma, melanoma, DLBCL, prostate cancer, cholangiocarcinoma, cervical cancer, AML, renal cell cancer, uveal melanoma, thyroid cancer, liposarcoma, HCC, or breast cancer.

In some embodiments, the solid cancer includes solid tumors that have an abnormal mass of tissue that may not contain cysts or liquid areas. Solid tumors may be benign or malignant. In some embodiments, examples of solid tumors include sarcomas, carcinomas, and lymphomas. In some embodiments, the solid cancer is carcinoma of the brain, kidney, liver, adrenal gland, bladder, breast, stomach, gastric tumors, ovaries, colon, rectum, prostate, pancreas, lung, vagina, cervix, testis, genitourinary tract, esophagus, larynx, skin, bone or thyroid, sarcoma, glioblastomas, neuroblastomas, gastrointestinal cancer, such as colon carcinoma or colorectal adenoma, a tumor of the neck and head, an epidermal hyperproliferation, prostate hyperplasia, a neoplasia, a neoplasia of epithelial character, adenoma, adenocarcinoma, keratoacanthoma, epidermoid carcinoma, large cell carcinoma, non-small-cell lung carcinoma, such as Hodgkin's and Non-Hodgkin's, a mammary carcinoma, follicular carcinoma, undifferentiated carcinoma, papillary carcinoma, seminoma, melanoma, an IL-1 driven disorder, an MyD88 driven disorder, or Smoldering of indolent multiple myeloma. In some embodiments, the hematological malignancy is a cancer that affects the blood, bone marrow, and lymph nodes. In some embodiments the hematological malignancy includes leukemias, lymphomas, and myelomas, such as acute lymphoblastic leukemia (ALL), chronic lymphocytic leukemia (CLL), large granular lymphocytic leukemia (LGL-L), B-cell prolymphocytic leukemia, acute myeloid leukemia (AML), Burkitt lymphoma/leukemia, primary effusion lymphoma, peripheral T-cell lymphoma (PTCL), cutaneous T-cell lymphoma (CTCL), diffuse large B-cell lymphoma (DLBCL), advanced B-cell diffuse large B-cell lymphoma (ABC DLBCL), intravascular large B-cell lymphoma, lymphoplasmacytic lymphoma, Waldenström's macroglobulinemia (WM), splenic marginal zone lymphoma, multiple myeloma, plasmacytoma, uveal melanoma, myelodysplastic syndrome (MDS), or myelodysplastic/myeloproliferative neoplasms (MDS/MPN).

In some embodiments the solid cancer or hematological malignancy is relapsed and/or refractory (R/R) high grade myeloid malignancies, acute lymphocytic leukemia (ALL), R/R lymphoma, or R/R solid tumors.

In some embodiments, the AML is caused by protein (e.g., of KMT2A or MLL) mutation or fusion. In some embodiments, the AML is a mutant or fusion protein AML, such as IDH1, DNMT3A, NPM1, ASXL1, FLT3-ITD, KMT2A-MLLT3, MLL-MLLT3, or MLL-AF9.

In some embodiment, the present disclosure provides a method of treating a benign proliferative disorder, such as, but are not limited to, benign soft tissue tumors, bone tumors, brain and spinal tumors, eyelid and orbital tumors, granuloma, lipoma, meningioma, multiple endocrine neoplasia, nasal polyps, pituitary tumors, prolactinoma, pseudotumor cerebri, seborrheic keratosis, stomach polyps, thyroid nodules, cystic neoplasms of the pancreas, hemangiomas, vocal cord nodules, polyps, and cysts, Castleman disease, chronic pilonidal disease, dermatofibroma, pilar cyst, pyogenic granuloma, and juvenile polyposis syndrome.

In some embodiments, the cancer is a leukemia, for example a leukemia selected from acute monocytic leukemia, acute myelogenous leukemia, chronic myelogenous leukemia, chronic lymphocytic leukemia and mixed lineage leukemia (MLL). In another embodiment the cancer is NUT-midline carcinoma. In another embodiment the cancer is multiple myeloma. In another embodiment the cancer is a lung cancer such as small cell lung cancer (SCLC). In another embodiment the cancer is a neuroblastoma. In another embodiment the cancer is Burkitts lymphoma. In another embodiment the cancer is cervical cancer. In another embodiment the cancer is esophageal cancer. In another embodiment the cancer is ovarian cancer. In another embodiment the cancer is colorectal cancer. In another embodiment, the cancer is prostate cancer. In another embodiment, the cancer is breast cancer.

In some embodiments, the present invention provides a method of treating triple negative breast cancer in a patient in need thereof, comprising administering Compound A of the present invention, or a pharmaceutically acceptable salt thereof.

In some embodiments, the present invention provides a method of treating acute lymphoblastic leukemia (ALL) in a patient in need thereof, comprising administering Compound A of the present invention, or a pharmaceutically acceptable salt thereof.

In some embodiments, the present invention provides a method of treating chronic lymphocytic leukemia (CLL), comprising administering Compound A of the present invention, or a pharmaceutically acceptable salt thereof.

In some embodiments, the present invention provides a method of treating large granular lymphocytic leukemia (LGL-L) in a patient in need thereof, comprising administering Compound A of the present invention, or a pharmaceutically acceptable salt thereof.

In some embodiments, the present invention provides a method of treating B-cell prolymphocytic leukemia, comprising administering Compound A of the present invention, or a pharmaceutically acceptable salt thereof.

In some embodiments, the present invention provides a method of treating acute myeloid leukemia (AML) in a patient in need thereof, comprising administering Compound A of the present invention, or a pharmaceutically acceptable salt thereof.

In some embodiments, the present invention provides a method of treating leukemia (e.g., AML) in a patient in need thereof, comprising administering Compound A of the present invention or a pharmaceutically acceptable salt thereof and a BCL-2 inhibitor (e.g., venetoclax). In some aspects of the method of treating leukemia (e.g., AML) with a combination of Compound A and a BCL-2 inhibitor (e.g., venetoclax), the combination is additive. In some aspects of the method of treating leukemia (e.g., AML) with a combination of Compound A and a BCL-2 inhibitor (e.g., venetoclax), the combination acts synergistically.

In some embodiments, the present invention provides a method of treating leukemia (e.g., AML) in a patient in need thereof, comprising administering Compound A of the present invention or a pharmaceutically acceptable salt thereof and a BCL-2 inhibitor (e.g., venetoclax), wherein the lymphoma is resistant to treatment (e.g., refractory) with the BCL-2 inhibitor (e.g., venetoclax) alone.

In some embodiments, Compound A of the present invention or a pharmaceutically acceptable salt thereof is administered to a patient at the doses and schedules provided herein and the BCL-2 inhibitor (e.g., venetoclax) is administered to the patient once every 1, 2, 3, 4, 5, 6, 7, 14, or 21 days.

In some embodiments, the BCL-2 inhibitor (e.g., venetoclax) is administered to the patient daily (QD). In some aspects, the BCL-2 inhibitor (e.g., venetoclax) is administered to the patient orally. In other aspects, the BCL-2 inhibitor (e.g., venetoclax) is administered to the patient at a dose of about 5 mg/kg to about 20 mg/kg (e.g., about 7 mg/kg, about 8 mg/kg, about 9 mg/kg, or about 10 mg/kg).

In some embodiments, the present invention provides a method of treating melanoma (e.g., uveal melanoma) comprising administering to a patient in need thereof Compound A or a pharmaceutically acceptable salt thereof and a BCL-2 inhibitor (e.g., venetoclax). In some aspects of the method of treating melanoma (e.g., uveal melanoma) with a combination of Compound A and a BCL-2 inhibitor (e.g., venetoclax), the combination is additive. In some aspects of the method of treating melanoma (e.g., uveal melanoma) with a combination of Compound A and a BCL-2 inhibitor (e.g., venetoclax), the combination acts synergistically.

In some embodiments, the present invention provides a method of treating melanoma (e.g., uveal melanoma) comprising administering to a patient in need thereof Compound A or a pharmaceutically acceptable salt thereof and a BCL-2 inhibitor (e.g., venetoclax), wherein the melanoma is resistant to treatment with the BCL-2 inhibitor (e.g., venetoclax) alone.

In some embodiments, the present invention provides a method of treating leukemia (e.g., AML) comprising administering to a patient in need thereof Compound A or a pharmaceutically acceptable salt thereof and a FLT3 inhibitor (e.g., midostaurin). In some aspects of the method of treating leukemia (e.g., AML) with a combination of Compound A and FLT3 inhibitor (e.g., midostaurin), the combination is additive. In some aspects of the method of treating leukemia (e.g., AML) with a combination of Compound A and a FLT3 inhibitor (e.g., midostaurin), the combination acts synergistically.

In some embodiments, the present invention provides a method of treating leukemia (e.g., AML) comprising administering to a patient in need thereof Compound A or a pharmaceutically acceptable salt thereof and a FLT3 inhibitor (e.g., midostaurin), wherein the lymphoma is resistant to treatment with the FLT3 inhibitor (e.g., midostaurin) alone.

In some embodiments, the present invention provides a method of treating a solid cancer comprising administering to a patient in need thereof Compound A or a pharmaceutically acceptable salt thereof and azacitidine.

In some embodiments, the present invention provides a method of treating a solid cancer comprising administering to a patient in need thereof Compound A or a pharmaceutically acceptable salt thereof and cytarabine.

In some embodiments, the present invention provides a method of treating a solid cancer comprising administering to a patient in need thereof a provided MDM2 degrader or a pharmaceutically acceptable salt thereof and a MEK inhibitor (e.g., selumetinib).

In some embodiments, Compound A of the present invention or a pharmaceutically acceptable salt thereof is administered to a patient at the doses and schedules provided herein and the MEK inhibitor (e.g., selumetinib) is administered to the patient once every 1, 2, 3, 4, 5, 6, 7, 14, or 21 days.

In some embodiments, the MEK inhibitor (e.g., selumetinib) is administered to the patient daily (QD). In some aspects, the MEK inhibitor (e.g., selumetinib) is administered to the patient orally. In other aspects, the MEK inhibitor (e.g., selumetinib) is administered to the patient at a dose of about 0.01 mg/kg to about 5 mg/kg (e.g., about 0.1 mg/kg, about 0.5 mg/kg, or about 1 mg/kg).

In some embodiments, the present invention provides a method of treating Burkitt lymphoma/leukemia in a patient in need thereof, comprising administering Compound A of the present invention, or a pharmaceutically acceptable salt thereof.

In some embodiments, the present invention provides a method of treating primary effusion lymphoma in a patient in need thereof, comprising administering Compound A of the present invention, or a pharmaceutically acceptable salt thereof.

In some embodiments, the present invention provides a method of treating peripheral T-cell lymphoma (PTCL) in a patient in need thereof, comprising administering Compound A of the present invention, or a pharmaceutically acceptable salt thereof.

In some embodiments, the present invention provides a method of treating cutaneous T-cell lymphoma (CTCL) in a patient in need thereof, comprising administering Compound A of the present invention, or a pharmaceutically acceptable salt thereof.

In some embodiments, the present invention provides a method of treating diffuse large B-cell lymphoma (DLBCL) in a patient in need thereof, comprising administering Compound A of the present invention, or a pharmaceutically acceptable salt thereof.

In some embodiments, the present invention provides a method of treating advanced B-cell diffuse large B-cell lymphoma (ABC DLBCL) in a patient in need thereof comprising administering Compound A of the present invention, or a pharmaceutically acceptable salt thereof.

In some embodiments, the present invention provides a method of treating intravascular large B-cell lymphoma in a patient in need thereof, comprising administering Compound A of the present invention, or a pharmaceutically acceptable salt thereof.

In some embodiments, the present invention provides a method of treating lymphoplasmacytic lymphoma in a patient in need thereof, comprising administering Compound A of the present invention, or a pharmaceutically acceptable salt thereof.

In some embodiments, the present invention provides a method of treating Waldenstrom's macroglobulinemia (WM) in a patient in need thereof, comprising administering Compound A of the present invention, or a pharmaceutically acceptable salt thereof.

In some embodiments, the present invention provides a method of treating splenic marginal zone lymphoma in a patient in need thereof, comprising administering Compound A of the present invention, or a pharmaceutically acceptable salt thereof.

In some embodiments, the present invention provides a method of treating multiple myeloma in a patient in need thereof, comprising administering Compound A of the present invention, or a pharmaceutically acceptable salt thereof.

In some embodiments, the present invention provides a method of treating plasmacytoma in a patient in need thereof, comprising administering Compound A of the present invention, or a pharmaceutically acceptable salt thereof.

In some embodiments, the present invention provides a method of treating myelodysplastic syndrome (MDS) in a patient in need thereof, comprising administering Compound A of the present invention, or a pharmaceutically acceptable salt thereof.

In some embodiments, the present invention provides a method of treating myelodysplastic/myeloproliferative neoplasms (MDS/MPN) in a patient in need thereof, comprising administering Compound A of the present invention, or a pharmaceutically acceptable salt thereof.

In some embodiments, the present invention provides a method of treating malignant peripheral nerve sheath tumors (MPNST) in a patient in need thereof, comprising administering Compound A of the present invention, or a pharmaceutically acceptable salt thereof.

In some embodiments, the present invention provides a method of treating pancreatic cancer in a patient in need thereof comprising administering Compound A of the present invention, or a pharmaceutically acceptable salt thereof.

In some embodiments, the present invention provides a method of treating primary CNS lymphomas in a patient in need thereof, comprising administering Compound A of the present invention, or a pharmaceutically acceptable salt thereof.

In some embodiments, the present invention provides a method of treating Hodgkin's lymphoma in a patient in need thereof, comprising administering Compound A of the present invention, or a pharmaceutically acceptable salt thereof.

In some embodiments, the present invention provides a method of treating primary cutaneous T-cell lymphoma in a patient in need thereof, comprising administering Compound A of the present invention, or a pharmaceutically acceptable salt thereof.

In some embodiments, the present invention provides a method of treating solid and liquid tumors in a patient in need thereof, comprising administering Compound A of the present invention, or a pharmaceutically acceptable salt thereof.

In some embodiments, the present invention provides a method of treating MYD88 mutant Waldenström macroglobulinemia in a patient in need thereof, comprising administering Compound A of the present invention, or a pharmaceutically acceptable salt thereof.

In some embodiments, the present invention provides a method of treating NSCLC in a patient in need thereof, comprising administering Compound A of the present invention, or a pharmaceutically acceptable salt thereof.

In some embodiments, the present invention provides a method of treating uveal melanoma in a patient in need thereof comprising administering Compound A of the present invention, or a pharmaceutically acceptable salt thereof.

In some embodiments, the present invention provides a method of treating Ewing sarcoma in a patient in need thereof, comprising administering Compound A of the present invention, or a pharmaceutically acceptable salt thereof.

In some embodiments, the present invention provides a method for the treatment of adult patients with a solid cancer or hematological malignancy who have received one prior therapy.

In some embodiments, the present invention provides a method for the treatment of adult patients with a solid cancer or hematological malignancy who have received two prior therapies.

In some embodiments, the present invention provides a method for the treatment of adult patients with a solid cancer or hematological malignancy who have received three prior therapies.

In some embodiments, the present invention provides a method for the treatment of adult patients with a solid cancer or hematological malignancy who have received at least one prior therapy.

In some embodiments, the present invention provides a method for the treatment of adult patients with a solid cancer or hematological malignancy who have received at least two prior therapies.

In some embodiments, the present invention provides a method for the treatment of adult patients with a solid cancer or hematological malignancy who have received at least three prior therapies.

In some embodiments, the present invention provides a method of increasing one or more protein markers (e.g., GFD15, p53, p21, and PUMA) or apoptotic markers (e.g., PHLDA3) in a patient in need thereof, comprising administering Compound A of the present invention, or a pharmaceutically acceptable salt thereof. In some embodiments, the method of increasing one or more protein markers (e.g., GFD15, p53, p21, and PUMA) or apoptotic markers (e.g., PHLDA3) comprises treating a solid cancer or hematological malignancy in a patient.

In some embodiments, the present invention provides a method of increasing one or more markers of p53 activity in a patient in need thereof, comprising administering Compound A of the present invention, or a pharmaceutically acceptable salt thereof. In some embodiments, the present invention provides a method of decreasing one or more markers of p53 activity in a patient in need thereof, comprising administering Compound A of the present invention, or a pharmaceutically acceptable salt thereof. In some embodiments, the one or more markers of p53 activity is a downstream target of p53 transcriptional activity. In some embodiments, the one or more markers of p53 activity is selected from MDM2, CDKN1A, TNFRSF10B, FAS, FASIPO8, BBC3, BAX, PHLDA2, PHLDA3, BCL2L1, BCL2, CASP8, TP53, TP53I3, PUM1, PARK7, GAPDH, MCL1, CRBN, ACTB, and ACTA2.

In some embodiments, the one or more markers of p53 activity is a p53 mRNA target encoded by MDM2, CDKN1A, TNFRSF10B, FAS, BBC3, BAX, PHLDA2, or TP53I3, as determined by RT-qPCR. In some embodiments, the one or more markers of p53 activity is a non-p53 mRNA target encoded by IPOS or PUM1, as determined by RT-qPCR. In some embodiments, the one or more markers of p53 activity is a p53 protein target encoded by MDM2, BCL2L1, BCL2, BAX, BBC3, CASP8, TP53, CDKN1A, PHLDA3, TP53I3, or FAS, as determined by mass spectrometry. In some embodiments, the one or more markers of p53 activity is a non-p53 protein target encoded by PARK7, GAPDH, MCL1, CRBN, ACTB, or ACTA2, as determined by mass spectrometry.

In some embodiments, the method of increasing one or more markers of p53 activity comprises treating a solid cancer or hematological malignancy in a patient. In some embodiments, the method of decreasing increasing one or more markers of p53 activity comprises treating a solid cancer or hematological malignancy in a patient.

### EXEMPLIFICATION

Compound A can be prepared by methods known to one of ordinary skill in the art, for example, as disclosed in WO 2021/188948, the contents of which are incorporated herein by reference in their entireties.

### Example 1. Pharmaceutical Development

### Drug Substance

Compound A was manufactured in free base form.

### Excipients

Polyethylene glycol 400, polysorbate 80, ethanol and butylated hydroxytoluene were used in the manufacture of the drug product. A liquid mixture of the above excipients was used to dissolve the drug substance. Both polyethylene glycol 400 and ethanol were used as the co-solvents, and polysorbate 80 was used as the surfactant to dissolve Compound A drug substance. Butylated hydroxytoluene was used as antioxidant to prevent polyethylene glycol 400 and polysorbate 80 from oxidation. All excipients met their respective pharmacopeial standards. The excipient selection and levels were based upon manufacturability, driven by solubility enhancement, stability, and functionality.

### Drug Product

### Formulation Development

Compound A for injection was manufactured as a concentrated frozen solution containing 10 mg/mL of free base intended to be diluted with an intravenous (IV) infusion vehicle (normal saline, 0.9% sodium chloride, injection). Compound A drug substance has poor aqueous solubility of less than 0.01 mg/mL at pH range between 3 to 8. The measured pKa and Log D of Compound A free base were 5.0 and >3.7, respectively. To achieve drug concentration of 10 mg/mL, formulation development focused on identifying acceptable pharmaceutical solvents, surfactants, and pH modifiers to achieve the required solubility of Compound A.

Initial solubility screening of Compound A focused on using different enabling excipients suitable for IV administration. The screening included surfactants, cosolvents, and pH modifiers (i.e., glacial acetic acid, and hydrochloric acid). The study indicated that the solubility of Compound A was significantly enhanced in formulations containing propylene glycol, polyethylene glycol 400, Solutol HS15 and polysorbate 80 at low pH. As a result, the selected formulation to support the GLP toxicology study was comprised of 30% propylene glycol, 3% Solutol HS15, and 0.01 M in acetate buffer at pH 5±0.2.

The clinical formulation development was centered on using organic concentrates without water to avoid hydrolysis of Compound A. Additional solubility and stability evaluations of Compound A were conducted with different types of co-solvents and surfactants with and without butylated hydroxytoluene as antioxidant. Thes studies revealed that combination of polyethylene glycol 400, ethanol, polysorbate 80 and butylated hydroxytoluene provide sufficient solubility and stability for Compound A at 10 mg/mL. Data from the development batch demonstrated that Compound A drug product remains stable through 1 month at the long-term storage condition of −20° C. and the accelerated condition of 5±3° C. with no significant changes in stability-indicating attributes as shown in Table 1 and Table 2.

TABLE 1
Stability of Drug Product at −20 ± 3° C.

| | | Time, months | |
|---|---|---|---|
| Attribute | Acceptance Criteria | Initial | 1 |
| Appearance | Clear, colorless to slightly yellow | Clear, pale | Clear, colorless solution |
| | solution that is essentially free of | yellow liquid | essentially free of visible |
| | visible particulate matter | | particulate matter |
| Assay | 90.0-110.0% (Lable Claim) | 97.1 | 95.9 |
| Total Impurities | ≤6.0 | 4.5 | 4.6 |
| (% area) | | | |
| pH | — | 5.3 | 5.8 |
| Particulate | ≥10 μm: ≤6000 particles per container | 1053 | 758 |
| matter | ≥25 μm: ≤600 particles per container | 22 | 5 |

TABLE 2
Stability of Drug Product at 5 ± 3° C.

| | | Time, months | |
|---|---|---|---|
| Attribute | Acceptance Criteria | Initial | 1 |
| Appearance | Clcar, colorless to slightly yellow | Clear, pale | Clcar, colorless solution |
| | solution that is essentially free of | yellow liquid | essentially free of visible |
| | visible particulate matter | | particulate matter |
| Assay | 90.0-110.0% (Lable Claim) | 97.1 | 96.8 |
| Total Impurities | ≤6.0 | 4.5 | 4.5 |
| (% area) | | | |
| pH | — | 5.3 | 5.8 |
| Particulate | ≥10 μm: ≤6000 particles per container | 1053 | 414 |
| matter | ≥25 μm: ≤600 particles per container | 22 | 4 |

### Description and Composition

The drug product, Compound A Injection (Concentrate Solution for Infusion), consists of a clear, colorless to slightly yellow solution of Compound A in clear glass vials with stopper and flip-off seal. The drug product was formulated as 10 mg/mL Compound A drug substance dissolved in an organic mixture containing polyethylene glycol 400, polysorbate 80, ethanol, and butylated hydroxytoluene.

The label fill volume was 5.0 mL. Each vial contains adequate sterile Compound A solution to deliver nominally 5.0 mL of the solution. The drug product solution is intended to be diluted to the required concentration with normal saline for intravenous infusion. The composition of the drug product is given in Table 3.

TABLE 3
Composition of Compound A for Injection

| | | | Amount |
|---|---|---|---|
| Component | Function | % w/w | per 5.0 mL |
| Compound A drug substance | Active | 0.93 | 0.05 g (active) |
| | ingredient | | |
| Polyethylene glycol 400 | Co-solvent | 69.34 | 3.72 g |
| Ethanol (200 proof) | Co-solvent | 9.91 | 0.53 g |
| Polysorbate 80 | Surfactant | 19.81 | 1.06 g |
| Butylated hydroxytoluene | Antioxidant | 0.01 | 0.0005 g |

### Manufacturing Process

The drug product was manufactured by preparing a solution of polyethylene glycol (PEG) 400, polysorbate 80, ethanol (200 proof), and butyl-hydroxytoluene. Compound A drug substance was dissolved in the organic solution at about 23° C. to achieve a concentration of 10 mg/mL.

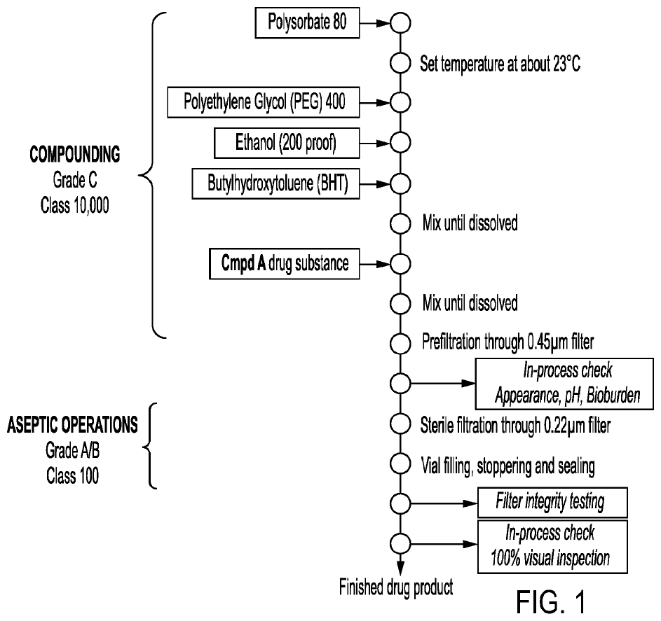

The prepared solution was first prefiltered through a 0.45 μm filter. After testing for appearance, pH and bioburden, the prefiltered solution was passed through two sterilizing filters (0.22 μm) in series to obtain a sterile solution. The sterile solution was filled into glass vials, stoppered, and crimped aseptically. Each vial was filled by weight to contain 5.0 mL of the sterile solution. The finished product was 100% visually inspected, and a post-sterile filtration filter integrity test was conducted. The vials were stored at −20° C. The flow diagram of the manufacturing process is shown in FIG. 1.

Frozen concentrated solutions of formulated Compound A were developed to be diluted in an IV vehicle before dosing intravenously through an administration kit. The compatibility of the formulated Compound A at 10 mg/mL was confirmed with 0.9/c sodium chloride (normal saline) based on an assessment of appearance, pH, assay, and impurity profile. The drug product was sterile filtered and filled into a 10 mL, Type 1 clear pre-sterilized borosilicated glass vials fitted with stopper with Flurotec barrier film secured by a flip-off seal.

### Injection Freeze-Thaw (FT) Study

To assess the stability of the thawed drug product, laboratory experiments were carried out under conditions representative of clinical dose preparation.

A freeze-thaw stability study was conducted for the frozen Compound A Injection, 10 mg/mL. Each FT cycle involved freezing the drug product vial at −20° C. for 24 hours followed by the complete thawing at room temperature. Three FT cycles were performed, with ten samples taken after each cycle (FT-1×, FT-2×, and FT-3×). All the vials were tested for appearance, assay, and impurities.

As presented in Table 4, the results show acceptable physicochemical stability at least up to 3 FT cycles. All stability-indicating parameters of FT-1×, FT-2× and FT-3× are comparable to the initial sample (FT-T0) and remained well within the product specifications at each FT cycle.

TABLE 4
Freeze-Thaw Cycle Study Data for Compound A Injection, 10 mg/mL

| | FT-T0 | FT-1x | FT-2x | FT-3x |
|---|---|---|---|---|
| Appearance of | Clear, slightly | Clear, slightly | Clear, slightly | Clear, slightly |
| Thawed Drug Product | yellow solution | yellow solution | yellow solution | yellow solution |
| Assay (% LC) | 97.8* | 97.7 | 98.0 | 96.5 |
| Total Impurities | 4.8 | 5.1 | 4.9 | 4.9 |
| (% area) | | | | |

*Sterile filtered sample was tested after 48 hours storage at room temperature

### Compound A Injection IV Dosing Solutions

Studies were conducted to assess the stability and compatibility of Compound A Injection IV dosing solutions with the IV administration supplies (bag, tubing and close system transfer device) that are intended for use in the clinical trials. Laboratory experiments were carried out under conditions representative of the clinical dose preparation. The IV bag, administration sets (tubing), and closed system transfer device (CSTD) that were utilized in this study are provided in Table 5.

TABLE 5
IV Administration Components Used in Stability and Compatibility
Studies of Compound A Injection Dosing Solutions

| IV Administration | Minimal Requirements of | Example Component Used in |
|---|---|---|
| Component | Component Used in Clinical Trial | Stability/Compatibility Study |
| 0.9% sodium chloride | Meet USP specification for 0.9% | B. Braun, Excel IV bags (Ethylene- |
| IV infusion bag (1000 | sodium chloride for Injection | Propylene copolymer). Product# L8000 |
| cc) | PVC/DEHP-free | |
| Closed System | Meet NIOSH guideline (DHHS | B. Braun On-Guard |
| Transfer System | Publication No. 2004-165 DEHP- | Vial adaptor (Product# 412111) |
| (CSTD) | free | Syringe adaptor (Product# 412118) |
| | | Spike port adaptor (Product# 412113) |
| | | General sterile luer lock syringe |
| | | (Becton Dickinson) |
| IV infusion set | No DEHP and not made with | B. Braun Infusomat space pump |
| | natural rubber | IV administrative set (Product# 490102) |
| Add on air elimination | No DEHP and not made with | B. Braun 1.2 μm air-eliminating filter |
| filter | natural rubber | (Product# 473994) |
| Catheter | PVC/DEHP-free and not made with | B. Braun Introcan Safety catheter |
| | natural rubber | (Product# 4251601-02) |

To accommodate a range of eventual clinical doses, in-use compatibility and stability studies were conducted at bracketing IV bag solution concentrations of 0.3 and 0.03 mg/ml, (300 and 30 mg equivalents in a 1000 cc bag). Once the drug product solution was added to the IV bag, the resulting diluted IV dosing solution was thoroughly mixed by hand and allowed to remain under ambient laboratory lighting and temperature conditions for the duration of the study. Samples were then pulled at 0, 4, and 24 hours from the IV bag port via syringe. These samples were subsequently tested for stability-indicating parameters, including appearance, pH, assay, and impurities. The in-use stability data showed that the IV dosing solutions at both ends of the concentration range (0.3 mg/mL and 0.03 mg/mL) are compatible with the IV bag and IV tubing and stable up to 24 hours (IV bag) and 8 hours (IV tubing) at ambient laboratory conditions. All stability-indicating parameters remained within the product specifications at each time point and showed no significant change.

### Conclusions

The data from these studies indicates that Compound A Injection solution, 10 mg/mL, shows acceptable physicochemical stability after 3 cycles of freezing and thawing. The drug product also shows acceptable stability under ambient storage conditions in the IV bag and IV infusion tubing for 24 hours and 8 hours, respectively. Moreover, the results from these studies suggest acceptable compatibility of Compound A injection with the intended diluent (0.9% sodium chloride) and containment/administration system (IV bag/tubing) to be used in the clinical setting.

### Example 2. A Phase 1, Multicenter, Open-Label, Dose-Escalation and Expansion Study to Evaluate the Safety, Tolerability, Pharmacokinetics, Pharmacodynamics, and Clinical Activity of Intravenously Administered Compound A in Adult Patients with Acute Myeloid Leukemia

Study Rationale: Targeted protein degraders represent a new therapeutic class of compounds that utilize the ubiquitin proteasome system to target the specific degradation of proteins. Compound A is a protein degrader that targets MDM2, a critical negative regulator of the tumor suppressor p53, that plays a key role in its transcriptional activity, protein stability, and nuclear localization. MDM2 expression is upregulated in many solid tumors, lymphomas, and acute leukemias resulting in loss of p53-dependent activities such as apoptosis and cell-cycle arrest.

### Objectives and Endpoints

### Phase 1a:

| Objectives | Endpoints |
|---|---|
| Primary | |
| To evaluate the safety and tolerability of escalating | Incidence and severity of adverse events (AEs) |
| doses of Compound A and to determine the | graded according to the National Cancer Institute |
| maximum tolerated dose (MTD)/maximum | (NCI) Common Terminology Criteria for Adverse |
| evaluated dose (MED) and the recommended | Events (CTCAE), version 5.0, clinical laboratory |
| Phase 2 dose (RP2D) separately in patients with | abnormalities, and electrocardiogram (ECG) |
| relapsed and/or refractory (R/R) acute myeloid | abnormalities defined as dose-limiting toxicities |
| leukemia (AML) and solid tumors | (DLTs) |
| Secondary | |
| To characterize the pharmacokinetics (PK) of | PK parameters derived from Compound A plasma |
| Compound A and any metabolites in plasma and | and urinc concentrations |
| urine | |
| To assess clinical activity of Compound A | As assessed in the following populations: |
| | Leukemias and HR-MDS: |
| | AML: CR, CR with partial hematologic recovery |
| | (CRh), Transfusion independence (TI), and |
| | OS, according to the International Working |
| | Group (IWG) (Cheson et al 2003) |
| | ALL: Hematological remission rate defined as CR |
| | and CRi |
| | MDS: CR, PR, Marrow CR and HI per IWG |
| | criteria |
| | Solid tumor malignancies: Overall response rate |
| | (ORR) defined as percentage of patients with |
| | complete response (CR) or partial response (PR) |
| | per RECIST 1.1 |
| To evaluate duration of response (DOR) in patients | As assessed in the following populations: |
| treated with Compound A | AML: Duration of CR/CRh (DoCR and DoRh) |
| | Solid tumor: DOR defined as time from the first |
| | response of PR or CR to death, progression, or last |
| | adequate assessment in all cohorts |
| Exploratory | |
| To evaluate the relationship between baseline | The relationship of MDM2 amplification, copy |
| MDM2 and p53 status and response to Compound A | number and P53 wild type status to Compound A |
| | response in tumor and ctDNA |
| To evaluate the relationship between baseline | Somatic mutation analysis of tumor tissue |
| tumor genotype and response to Compound A | |
| To assess the pharmacodynamic (PD) effects of | PD changes in both peripheral blood mononuclear |
| Compound A | cells (PBMCs) and tumor tissues between pre- and |
| | post-dose sample collections, as applicable |
| To assess possible PK/PD and PK/efficacy | PK/PD and PK/efficacy correlations from PBMC |
| correlations | and tissue samples, as applicable |

### Phase 1b:

| Objectives | Endpoints |
|---|---|
| Primary | |
| To evaluate the safety and tolerability of | Incidence and severity of AEs graded according |
| Compound A at the RP2D in patients with R/R | CTCAE, version 5.0, and changes in clinical |
| AML | laboratory parameters, vital signs, and ECGs |
| Secondary | |
| To obtain preliminary estimates of clinical activity | Leukemias: |
| of Compound A in adult patients with AML | AML- ORR according to the International |
| | Working Group (IWG) |
| To evaluate DOR in adult patients in all cohorts | DOR defined as time of initial response until |
| | documented tumor progression, death, or last |
| | adequate assessment. |
| To evaluate progression-free survival (PFS) | |
| To evaluate overall survival (OS) in all cohorts. | OS defined as time from treatment initiation to |
| | death from any cause. |
| To characterize the pharmacokinetics (PK) of | PK parameters derived from of Compound A |
| Compound A and any metabolites in plasma and | plasma and urinc concentrations |
| urine | |
| Exploratory | |
| To evaluate the relationship between baseline | Somatic mutation analysis of tumor tissue |
| tumor genotype and response to of Compound A | |
| To assess the pharmacodynamic (PD) effects of | Protein analysis of tumor tissue and peripheral |
| Compound A in adult patients treated with | blood mononuclear cells (PBMCs) in pre and post |
| Compound A | treatment samples |
| To explore the pharmacokinetics (PK) and PD | PK/PD correlation between tumor and PBMCs |
| relationship of Compound A | PD changes in both plasma and tumor tissues |
| | between pre- and post-dose collections |

### Overall Design

The first-in-human study of Compound Ais an open-label Phase 1a (dose escalation)/1b (dose expansion) study in adult patients with advanced hematological malignancies and advanced/metastatic solid tumors.

The Phase 1a will be initiated in patients with advanced hematological malignancies and advanced solid tumors (Phase 1a-Cohort 1). Patients with a known p53^{WT} status will have enrollment priority during the Phase 1a. When a sufficient number of patients have been treated to provide a preliminary assessment of the toxicity profile of Compound A, including bone marrow toxicity (i.e., incidence of ≥grade 3 neutropenia or decreased platelets), a separate dose-escalation cohort will be initiated in patients with advanced acute leukemias and high risk MDS (Phase 1a-Cohort 2). Initiation of Phase 1a, Cohort 2, Dose Level 1 and the starting dose level will be determined by the SRC following review of the totality of the safety and available pharmacokinetics and pharmacodynamics data. Cohorts 1 and 2 will continue dose escalation separately based on a Bayesian Optimal Interval Design (BOIN).

Once the MTD/RP2D have been determined in Cohort 2, the leukemia phase 1b expansion will be initiated in up to 20 patients with R/R AML. In phase 1a, tumor p53 status (mutant or wild-type) will not be required and may be assessed retrospectively. Patients with advanced solid tumor and wild-type p53 (p53^{WT}) status will be prioritized during the Phase 1a. In phase 1b, p53 status will be required (locally or centrally assessed) prior to dosing. If assessed locally, confirmatory testing will be performed retrospectively from archival tumor. Solid tumor indications for the Phase 1b will be determined after initiation of the study and outlined in a protocol amendment.

Patients who provide informed consent and meet the eligibility criteria for the study will be enrolled and will be treated with Compound A once every 3 weeks until disease progression, unacceptable toxicity, withdrawal of consent, any study-specific discontinuation criteria are met, or the Investigator determines that it is in the best interest of the patient to discontinue study treatment. The end of treatment/safety follow-up visit will be scheduled within 30 days from the last dose of Compound A and prior to initiation of a new anticancer therapy, whichever occurs first. Further, patients will be contacted every 3 months to collect data on survival status and subsequent therapies for up to one year after their last dose of Compound A.

When archival tissue/slides/blocks are not available, pre-dose biopsy will be performed (optional for Phase 1a, required for Phase 1b). Another on-treatment biopsy will be required in Phase 1b unless medically contraindicated or is unattainable due to lack of feasibility. An additional biopsy at time of disease progression will be optional for all patients. Any issues with collection of biopsies are to be discussed with medical monitor.

Up to 30-40 evaluable patients will be enrolled in each Cohort in Phase 1a; the total number of patients will depend on the number of dose levels explored. Up to 20 patients will be enrolled in the Phase 1b.

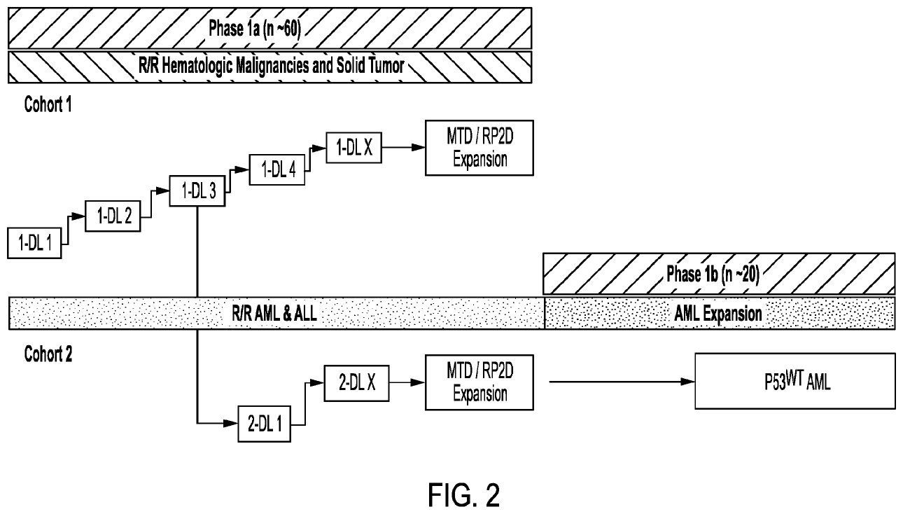

The study schema is provided in FIG. 2.

### Patient Eligibility for Solid Tumor Phase 1a

### Inclusion Criteria

1. Male or female aged ≥18 years on the day of signing informed consent.
2. Patient understands signed and dated, written informed consent and provides voluntary consent prior to any mandatory study-specific procedures, sampling, and analyses.
3. Histologically or pathologically confirmed solid tumor.
4. Fresh or archival formalin fixed paraffin embedded (FFPE) tumor tissue or 15 slides preferably collected within ideally 6 months prior to first dose of the study drug. When archival tissue/slides/blocks are not available, pre-dose biopsy will be performed.
5. Relapsed and/or refractory disease to at least two prior standard of care treatment or indications for whom standard therapies are not available.
6. At least one bi-dimensionally measurable disease site. The lesion must have a greatest transverse diameter of at least 1.5 cm and greatest perpendicular diameter of at least 1.0 cm at baseline.
7. Eastern Cooperative Oncology Group (ECOG) performance status 0-2.
8. Adequate organ function at Screening defined as:
   a) Absolute neutrophil count (ANC) ≥1000/μL.
   b) Hemoglobin ≥8 g/dL (for those patients undergoing red blood cell [RBC]transfusion, hemoglobin must be evaluated after at least 14 days after the last RBC transfusion).
   c) Platelet count ≥100,000/μL (assessed ≥7 days following last platelet transfusion in patients with thrombocytopenia requiring platelets).
   d) Aspartate aminotransferase (AST), alanine transaminase (ALT)≤3× upper limit of normal (ULN) or <5×ULN in cases of documented liver metastases.
   e) Total serum bilirubin ≤2.5×ULN or <5×ULN if secondary to Gilbert's syndrome or documented liver metastases.
   f) Serum creatinine clearance ≥60 mL/min either measured or calculated using standard Cockcroft-Gault formula).
9. Women of child-bearing potential (WOCBP) must agree to use highly effective contraceptive methods for the duration of study treatment and 6 months after the last dose of study medication (definitions for highly effective contraception will be included in the protocol).
10. WOCBP must have a negative serum pregnancy test at screening and a negative serum or urine pregnancy test within 72 hours prior to first dose.
11. Men must agree to use highly effective contraceptive methods during the study treatment and for 6 months after the last dose of study treatment if the partner is a WOCBP.

### Exclusion Criteria

1. History or suspicion of central nervous system (CNS) metastases.
2. History of or active concurrent malignancy unless the patient has been disease-free for ≥2 years. Exceptions to the ≥2-year time limit include treated basal cell or localized squamous cell skin carcinoma, localized prostate cancer, or other localized carcinomas such as carcinoma in situ of cervix, breast, or bladder.
3. Patient has not recovered from any clinically significant AEs of previous treatments to pre-treatment baseline or Grade 1 prior to first dose of study drug.
4. Ongoing unstable cardiovascular function:
   a) Symptomatic ischemia, or
   b) Uncontrolled clinically significant conduction abnormalities (i.e., ventricular tachycardia on anti-arrhythmic drugs is excluded; 1° degree atrioventricular block or asymptomatic left anterior fascicular block/right bundle branch block will not be excluded), or
   c) Congestive heart failure of New York Heart Association Class ≥III, or
   d) Myocardial infarction within 3 months prior to Screening.
   e) Congenital long QT syndrome, or a QT interval corrected by Fridericia's formula (QTcF) ≥450 ms (average of triplicate electrocardiograms) at Screening and/or on C1D1 (pre-dose) with the exception of a documented bundle branch block or unless secondary to pacemaker. In the case of a documented bundle branch block or a pacemaker, discussion with the Medical Monitor is required prior to enrollment.
5. History of thromboembolic or cerebrovascular event (i.e., transient ischemic attacks, cerebrovascular accidents, pulmonary emboli, or clinically significant deep vein thrombosis) within 1 year prior to Screening.
6. Subjects with an active severe infection that required anti-infective therapy or with an unexplained fever >38.5° C. during screening visits or on their first day of study drug administration (at the discretion of the investigator, subjects with tumor fever may be enrolled)
7. Positive hepatitis B and/or hepatitis C serology or known seropositivity for human immunodeficiency virus (HIV).
8. Positive severe acute respiratory syndrome coronavirus-2 (SARS-CoV-2) test at Screening.
9. Concurrent medical conditions including psychiatric disorders that in the judgment of the Investigator will interfere with the patient's ability to participate or with achieving the objectives of the study or pose a safety risk.
10. Patient is pregnant or breast feeding.
11. Radiation treatment within 4 weeks prior to first dose of study drug.
12. Major surgery requiring general anesthesia within 4 weeks prior to first dose of study drug. If patient required general anesthesia within the prior 4 weeks, consultation with the Medical Monitor is required prior to enrollment.
13. Received live vaccine within 1 month prior to the first dose of study drug.
14. Exposure to investigational or non-investigational anti-cancer therapy within 4 weeks or within at least 5 half-lives (up to a maximum of 4 weeks) prior to the first dose of study drug, whichever is longer. In all situations, the maximum washout period will not exceed 4 weeks prior to first dose of study drug.
   a) Note: Low dose steroids (oral prednisone or equivalent ≤20 mg/day), localized non-CNS radiotherapy, previous hormonal therapy with luteinizing hormone-releasing hormone (LHRH) agonists for prostate cancer, and treatment with bisphosphonates and RANKL inhibitors are not criteria for exclusion.
15. Patient has completed a course of SARS-CoV-2 vaccine within 14 days prior to first dose of study drug.
16. Patient is unable or unwilling to discontinue prohibited concomitant medications or adhere to restrictions for use of concomitant medications to be further defined in the protocol.
17. Patient is unable or unwilling to comply with all requirements of the study.
18. Person who has been committed to an institution by official or judicial order.
19. Sponsor or Investigator site staff who are directly involved in the conduct of the study, site staff otherwise supervised by the investigator, and their respective family members.

### Patient Eligibility for Leukemia Cohorts

### Inclusion Criteria

1. Primary diagnosis
   a) Phase 1a:
      i) AML: based on WHO classification that is Relapsed or Refractory to standard therapy or for which no standard therapies are anticipated to result in a durable remission or patients who are unsuitable to receive standard therapy due to age, performance status (PS), and/or adverse risk factors, according to the treating physician and with approval of the Medical Monitor.
      ii) ALL: based on/classification patients with Relapsed or Refractory disease who are due to receive either salvage 1 or salvage 2 therapy. Ph+ ALL patients must have failed at least 1 second generation tyrosine kinase inhibitor.
      iii) Relapsed/progressed high/very high risk MDS: (score ≥4.5) according to IPSS-R risk stratification, or subject that is intolerant to established therapy known to provide clinical benefit for their condition (i.e., subjects must not be candidates for regimens known to provide clinical benefit), according to the treating physician and with approval of the Medical Monitor.
   b) Phase 1a and 1b:
      i) ALL patients: bone marrow involvement ≥5% lymphoblasts.
   c) Phase 1b:
      i) Relapsed and/or Refractory AML defined as:
         (1) Subjects who relapsed after transplantation;
         (2) Subjects in second or later relapse;
         (3) Subjects who are refractory to initial induction or reinduction treatment;
         (4) Subjects who relapsed within 1 year of initial treatment.
      ii) AML patients:
         (1) Hydroxyurea allowed prior to Day 1 to keep peripheral blast count ≤10,000/μL; but must be discontinued 24 hours prior to first dose.
         (2) Subjects must have been off previous anti leukemia therapy for at least 2 weeks or 5 half-lives, whichever is shorter if the immediate prior regimen included only weekly chemotherapy; or 4 weeks from any therapy with therapeutic biologics and from any type of investigational therapy prior to the first dose.
2. Life expectancy ≥12 weeks.
3. Age ≥18 years old.
4. ECOG performance status: 0-2.
5. Resolved acute effects of any prior therapy to baseline severity or Grade ≤1 CTCAE except for AEs not constituting a safety risk by investigator judgment.
6. At least 4 weeks since radiotherapy prior to the first dose of Compound A. Patients must have passed nadir white blood cell (WBC) and platelet counts, have full recovery or stabilization of absolute neutrophil counts (ANC) and platelet counts, and ANC counts must have recovered from prior toxicity.
7. Adequate organ function at Screening defined as:
   a) Aspartate aminotransferase (AST), alanine transaminase (ALT)≤3× upper limit of normal (ULN), unless considered due to leukemic disease.
   b) Total serum bilirubin ≤1.5×ULN unless considered secondary to Gilbert's syndrome or leukemic disease.
   c) Serum creatinine clearance ≥50 mL/min either measured or calculated using standard Cockcroft-Gault formula).
   d) Platelet count ≥20,000/uL (transfusions to achieve this level are allowed). Subjects with a baseline platelet count of <20,000/uL due to underlying malignancy are eligible with Medical Monitor approval.
8. Women of child-bearing potential (WOCBP) must agree to use highly effective contraceptive methods for the duration of study treatment and 6 months after the last dose of study medication (definitions for highly effective contraception will be included in the protocol).
9. WOCBP must have a negative serum pregnancy test at screening and a negative serum or urine pregnancy test within 72 hours prior to first dose.
10. Men must agree to use highly effective contraceptive methods during the study treatment and for 6 months after the last dose of study treatment if the partner is a WOCBP.
11. Subjects must be amenable to serial bone marrow sampling, peripheral blood sampling, and urine sampling during study. The diagnosis and evaluation of Acute leukemia or MDS will be made by BM aspiration and biopsy. If an aspirate is unobtainable (i.e., a “dry tap”), the diagnosis may be made from core biopsy.
12. Documented TP53^{WT} disease (ONLY for patients in the Phase 1b).
13. Patient must be able to understand and willing to sign an informed consent. A legally authorized representative may consent on behalf of a subject who is otherwise unable to provide informed consent, if acceptable to and approved by the site and/or site's Institutional Review Board (IRB).

### Exclusion Criteria

1. AML patients:
   a) Diagnosis of acute promyelocytic leukemia (APL), AML with known central nervous system (CNS) involvement unless the patient has completed treatment for the CNS disease, has recovered from the acute effects of therapy prior to study entry, and is neurologically stable.
2. ALL patients:
   a) Isolated extramedullary relapse.
   b) Burkitt's or mixed lineage leukemia.
3. Active CNS leukemia. Patients with symptoms of CNS disease require a lumbar puncture to rule out CNS disease.
4. Prior chemotherapy within ≤2 weeks of first dose of Compound A with the following exceptions:
5. Steroids, hydroxyurea, oral mercaptopurine, methotrexate, vincristine, thioguanine, and tyrosine kinase inhibitors are permitted within 2 weeks of the first dose of Compound A as maintenance or to reduce the peripheral blast count.
6. Craniospinal radiation is prohibited; however, other concurrent therapy for CNS prophylaxis or treatment of CNS relapse is permitted.
7. Peripheral lymphoblasts >10,000/μL (treatment with hydroxurea and/or steroids is permitted within 2 weeks of first dose of Compound A to reduce the WBC count.
8. Known systemic vasculitides (e.g., Wegener's granulomatosis, polyarteritis nodosa, systemic lupus erythematosus), primary or secondary immunodeficiency (such as HIV infection or severe inflammatory disease.
9. Chronic graft versus host disease (GVHD), active GVHD with other than Grade 1 skin involvement, or GVHD requiring immunosuppressive treatment. Patients with GVHD receiving immunosuppressive treatment must stop the therapy at least 2 weeks prior to the first dose of Compound A.
10. Patient is known to be refractory to platelet or packed red cell transfusions per institutional guidelines.
11. Patient is within 3 months post allogenic hematopoietic stem cell transplant or within 30 days post autologous stem cell transplant, and the patient has not recovered from transplant-associated toxicities prior to the first dose of Compound A.
12. Subjects with an active severe infection that required anti-infective therapy or with an unexplained fever >38.5° C. during screening visits or on their first day of study drug administration (at the discretion of the investigator, subjects with tumor fever may be enrolled).
13. Evidence of uncontrolled current serious infection (including sepsis, bacteremia, fungemia, or history (within 4 months) of deep tissue infections such as fasciitis or osteomyelitis.
14. Current or chronic hepatitis B or C infection as evidenced by hepatitis B surface antigen and anti-hepatitis C antibody positivity, respectively, or known seropositivity for human immunodeficiency virus (HIV).
15. Participation in other studies involving investigational drug(s) within 4 weeks prior to the first dose of Compound A.
16. Major surgery within 4 weeks of study entry.
17. Chronic systemic corticosteroid treatment. Topical applications, inhaled sprays, eye drops, local injections of corticosteroids and systemic steroids required for acute medical interventions are allowed.
18. Other severe acute or chronic medical or psychiatric condition, including recent (within the past year) or active suicidal ideation or behavior, or laboratory abnormality that may increase the risk associated with study participation or investigational product administration or may interfere with the interpretation of study results and, in the judgment of the investigator, would make the patient inappropriate for entry into this study.
19. Patients who are investigational site staff members directly involved in the conduct of the study and their family members, site staff members otherwise supervised by the investigator, or patients who are Kymera employees directly involved in the conduct of the study.
20. Pregnant females; breastfeeding females; male patients with partners currently pregnant; male patients able to father children and female patients of childbearing potential who are unwilling or unable to use 2 highly effective methods of contraception as outlined in this protocol for the duration of the study and for at least 60 days after last dose of investigational product.
21. Any of the following in the previous 6 months: myocardial infarction, congenital long QT syndrome, Torsades de pointes, arrhythmias (including sustained ventricular tachyarrhythmia and ventricular fibrillation), right bundle branch block and left anterior hemiblock (bifascicular block), unstable angina, coronary/peripheral artery bypass graft, symptomatic congestive heart failure (CHF New York Heart Association class III or IV), cerebrovascular accident, transient ischemic attack, or symptomatic pulmonary embolism.
22. Ongoing cardiac dysrhythmias of NCI CTCAE Grade 22 or QTcF interval >470 msec (based on average of 3 consecutive ECGs) at screening.
23. Left ventricular ejection fraction (LVEF)<40% by echocardiogram (ECHO) or multi gated acquisition (MUGA) scan.
24. History of chronic liver disease or suspected alcohol abuse.
25. History of hepatic veno-occlusive disease (VOD) or sinusoidal obstruction syndrome (SOS).
26. Patient is unable or unwilling to discontinue prohibited concomitant medications or adhere to restrictions for use of concomitant medications to be further defined in the protocol.
27. Patient is unable or unwilling to comply with all requirements of the study.
28. Person who has been committed to an institution by official or judicial order.
29. Sponsor or Investigator site staff who are directly involved in the conduct of the study, site staff otherwise supervised by the investigator, and their respective family members.
30. Known hypersensitivity to any of the components of Compound A.

### Example 3: A Phase 1, Multicenter, Open-Label, Dose-Escalation Study to Evaluate the Safety, Tolerability, Pharmacokinetics, Pharmacodynamics, and Clinical Activity of Intravenously Administered Compound A in Adult Patients with High Grade Myeloid Malignancies and Acute Lymphocytic Leukemia, Lymphoma and Advanced Solid Tumors

This Phase 1 study will evaluate the safety, tolerability, pharmacokinetics/pharmacodynamics (PK/PD), and clinical activity of Compound A in adult patients with relapsed and/or refractory (R/R) high grade myeloid malignancies, acute lymphocytic leukemia (ALL), R/R lymphoma, and R/R solid tumors. The study will identify the pharmacologically optimal dose(s) of Compound A as the recommended Phase 2 dose (RP2D), based on all safety, PK, PD, and efficacy data.

This is an open-label Phase 1 (dose escalation) first-in-human study of Compound A in adult patients. This study will be initiated in patients with advanced high-grade myeloid malignancies, ALL, lymphomas, and solid tumors and will be comprised of two arms to characterize the safety and tolerability of ascending doses of Compound A in each arm. Arm A will consist of patients with lymphomas and advanced solid tumors and Arm B will consist of patients with high grade myeloid malignancies and ALL.

### Arms and Interventions

| Arms | Assigned Interventions |
|---|---|
| Experimental: Phase 1 Dose Escalation Arm A in | Compound A will be supplied as 10 mg/mL of frozen |
| patients with R/R Solid Tumors and Lymphomas | solution to be administered intravenously per the |
| Compound A dosed intravenous (IV) once every | defined protocol frequency and dose level. |
| three weeks in 21-day cycles | |
| Experimental: Phase 1 Dose Escalation Arm B in | Compound A will be supplied as 10 mg/mL of frozen |
| patients with R/R High Grade Myeloid | solution to be administered intravenously per the |
| Malignancies and ALL | defined protocol frequency and dose level. |
| Compound A dosed IV once every three weeks in | |
| 21-day cycles | |

### Outcome Measures

### Primary Outcome Measure:

1. Incidence and severity of adverse events. Adverse events graded according to National Cancer Institute Common Terminology Criteria for Adverse Events (NCI CTCAE), version 5.0

### Secondary Outcome Measure:

2. Area under the Plasma Concentration versus Time Curve (AUC) of Compound A. To determine the AUC from plasma concentrations in patients. Time Frame: Blood samples for PK analysis collected up to Day 15 during cycle 1 and cycle 2 (each cycle is 21 days).
3. Maximum Plasma Concentration of Compound A (Cmax). To determine the Cmax from plasma concentrations in patients. Time Frame: Blood samples for PK analysis collected up to Day 15 during cycle 1 and cycle 2 (each cycle is 21 days).
4. Time to maximum plasma concentration of Compound A (Tmax). To determine the Tmax from plasma concentrations in patients. Time Frame: Blood samples for PK analysis collected up to Day 15 during cycle 1 and cycle 2 (each cycle is 21 days).
5. Evidence of Clinical activity of Compound A in (R/R) High grade myeloid malignancies and ALL patients. Clinical activity of Compound A will be assessed according to the following response criteria: International Working Group (IWG) (Cheson, 2003) for AML; NCCN guidelines (2022) for ALL; IWG criteria (Cheson, 2006) for high risk MDS and MDS/MPN IWG (Savona, 2015) for MDS/MPN. Time Frame: From the time of the first dose of study drug through 30 days after the last dose of study drug or until disease recurrence or death, whichever occurs first, about 18 months.
6. Evidence of Clinical activity of Compound A in R/R Lymphoma patients. Objective Response Rate (ORR) based on Investigator's assessment as per Lugano criteria 2014 for Lymphomas. Time Frame: From Baseline scan until first documented progression or death from any cause, whichever comes first, about 18 months.
7. Evidence of Clinical activity of Compound A in R/R Solid Tumor patients. Overall response rate (ORR) defined as percentage of patients with CR or PR per RECIST 1.1. Time Frame: From Baseline scan until first documented progression or death from any cause, whichever comes first, about 18 months.
8. Duration of Response (DOR) in Patients Treated with Compound A. Duration of Response (DOR) in R/R high grade myeloid malignancies and ALL, lymphoma and solid tumor patients treated with Compound A. Time Frame: From date of first of response to the date of documented first progression or death whichever comes first, about 18 months.

### Inclusion Criteria

1. All Patients:
   a) Eastern Cooperative Oncology Group performance status: 0-2.
   b) Resolved acute effects of any prior therapy to baseline severity or Grade ≤1 NCI CTCAE
   c) Adequate organ and bone marrow function in the absence of growth factors
2. Solid Tumors and Lymphoma (Arm A) ONLY
   a) Histologically or pathologically confirmed solid tumor or lymphoma.
   b) Relapsed and/or refractory (R/R) disease to at least two prior standard-of-care treatments or tumors for whom standard therapies are not available.
3. Advanced high grade myeloid malignancies, and Acute Lymphocytic Leukemia (Arm B) ONLY
   a) Primary diagnosis of AML, ALL, Relapsed/progressed high-risk Myelodysplastic Syndromes (MDS), Myelodysplastic/myeloproliferative neoplasms (MDS/MPN). Must be R/R to standard therapies.
   b) At least 4 weeks since radiotherapy prior to the first dose of study drug.

### Exclusion Criteria:

1. All Participants:
   a) Ongoing unstable cardiovascular function.
   b) Major surgery within 4 weeks of study entry.
   c) History of or active concurrent malignancy unless disease-free for ≥2 years.
   d) Exposures to anticancer therapy within 2 weeks or 5 half-lives whichever is shorter; or 4 weeks from any biologics/immunotherapies or any investigational therapy prior to the first dose of study drug.
2. Solid Tumors and Lymphoma (Arm A) ONLY
   a) Known active uncontrolled or symptomatic central nervous system (CNS) metastases.
   b) Autologous hematopoietic stem cell transplant (HSCT) within six months prior to first dose of study drug or participant has progressed within six months from the day of stem cell infusion (for lymphoma participants only).
   c) Prior allogeneic hematopoietic stem cell transplant.
3. Advanced high grade myeloid malignancies, and ALL (Arm B) ONLY
   a) Active CNS leukemia. Participants with symptoms suggestive of CNS disease will require a lumbar puncture to rule out CNS disease.
   b) Prior chemotherapy/radiation within ≤2 weeks of first dose of study drug.
   c) Known systemic vasculitides (e.g., Wegener's granulomatosis, polyarteritis nodosa, systemic lupus erythematosus).
   d) Participant is within 3 months post allogenic hematopoietic stem cell transplant or within 30 days post autologous stem cell transplant, and the participant has not recovered from transplant-associated toxicities.
   e) Patients with active or chronic graft versus host disease (GVHD) or on treatment for GVHD.

### Example 4. Additional In Vitro and In Vivo Experiments

### In Vitro Assays

All cell lines were cultured according to recommended procedures unless otherwise noted. For growth inhibition assays, cells were treated with compounds for indicated time points. Viability was assessed using Promega® CellTiter-Glo® assay, and apoptosis was assessed using Promega® Caspase-Glo® 3/7 assay. Cell cycle arrest and/or apoptosis in response to single agent or combination treatment was assessed using flow cytometry analysis on cells treated for 24h.

### In Vivo Experiments

AML patient cells from leukapheresis were intravenously (IV) injected and established in immunocompromised host strain mice. Surrogate animals were used to determine the level of engraftment targeting a threshold of ˜20% huCD45+ cells in BM. Mice were randomized on study and treatment was initiated. Vehicle and Compound A, 1 mg/kg were administered IV, every three weeks for six weeks (total of two doses of Compound A). At study end, whole blood, bone marrow and spleen were assessed by flow cytometry.

RS4;11 and MV4;11 tumors were established subcutaneously in the hind flank of NOD/SCID mice. Mice were randomized and treated with Compound A as either a single pulse dose or weekly for 3 weeks at doses matched for total AUC. MDM2 SMI DS-3032 (Milademetan) was dosed at its clinically equivalent dose and regimen of 3 days on/11 days off. Pharmacodynamic effects were assessed at mRNA level by RT-qPCR and protein level by quantitative targeted mass spectrometry and immunohistochemistry.

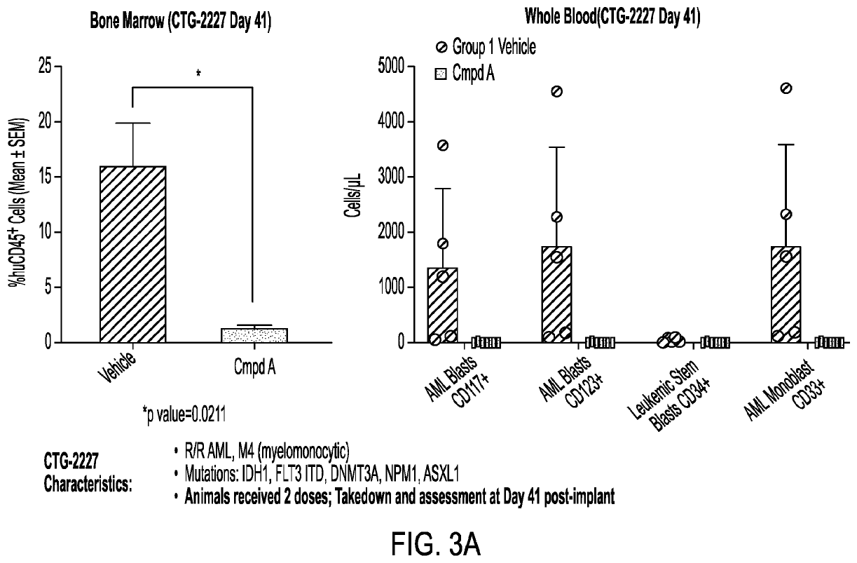

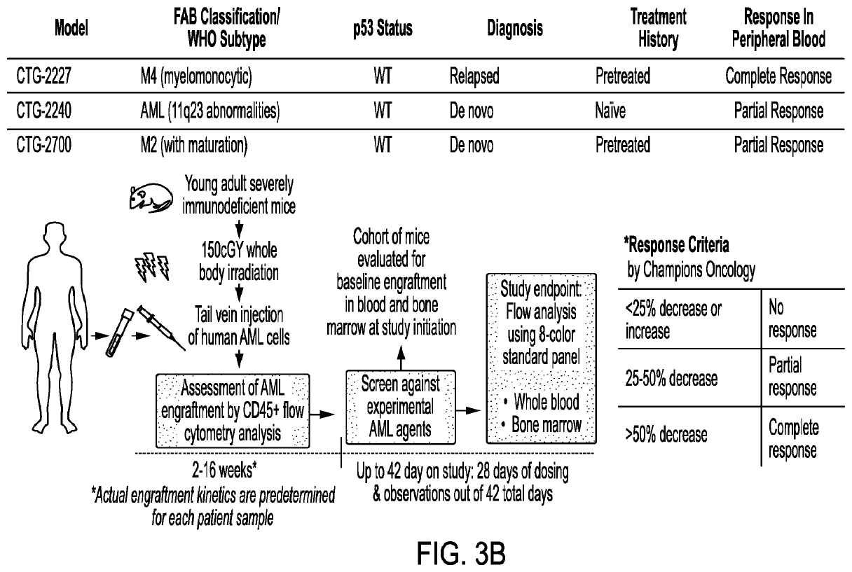

FIGS. 3A and 3B shows that Compound A (1 mg/kg, Q3W) achieves tumor regression in a CTG-2227 AML patient-derived xenograft (PDX) model and partial responses in CTG-2240 and CTG-2700 AML PDX models. Compound A significantly reduces hCD45+ cells in bone marrow and AML blasts.

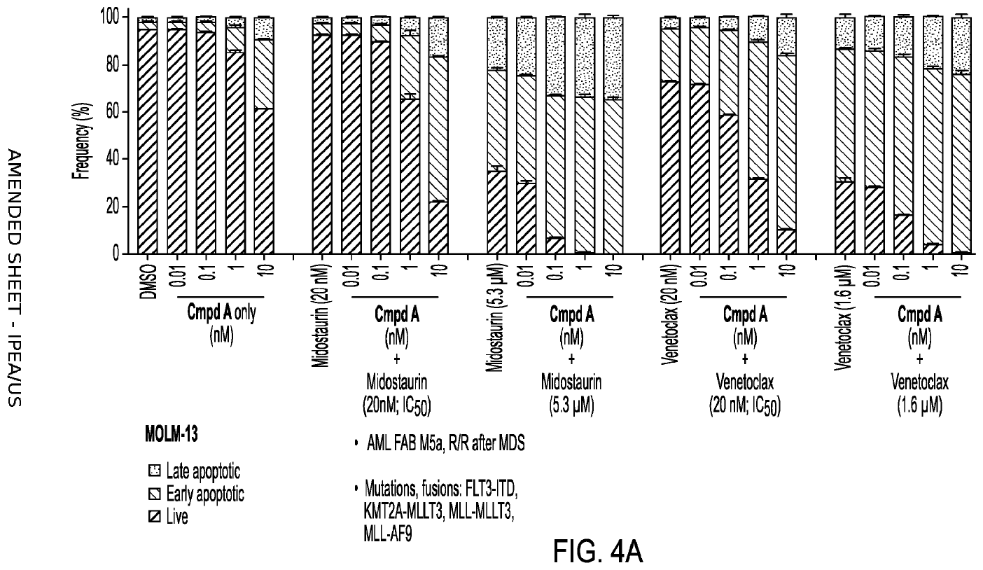

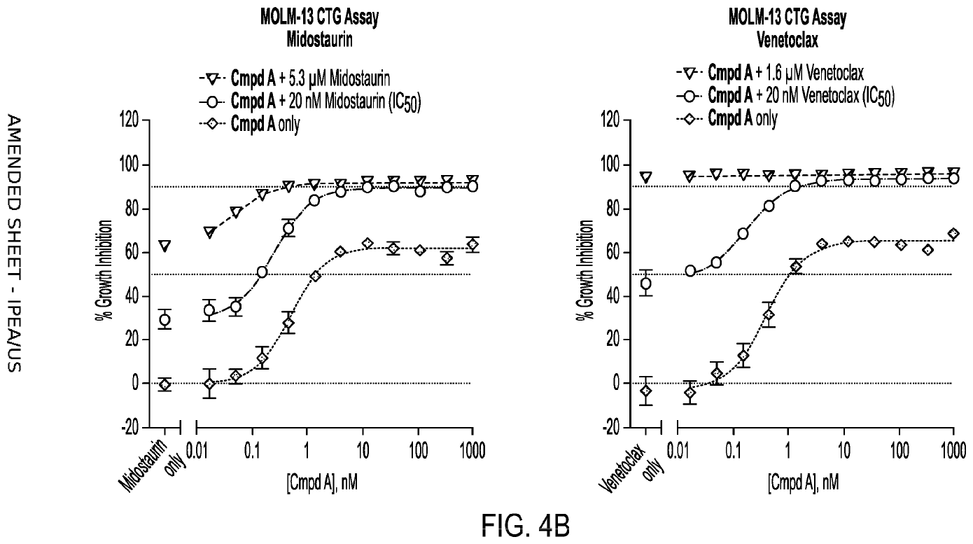

FIGS. 4A and 4B shows the combinatorial benefit of Compound A with venetoclax and midostaurin in MOLM-13 cell line. The data shows that Compound A combination with venetoclax and midostaurin enhances induction of apoptosis and cell killing in MOLM-12 AML cell line.

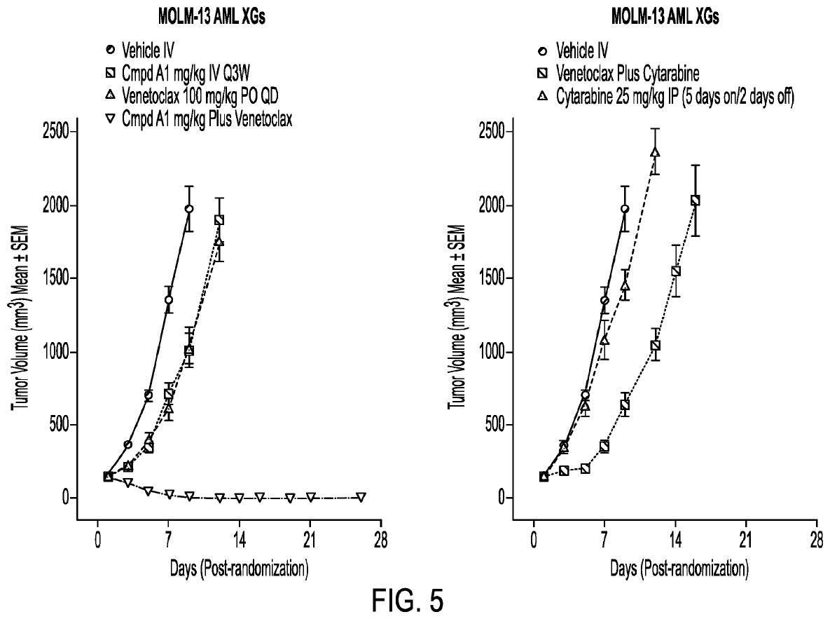

FIG. 5 shows the significant combinatorial benefit of Compound A with standard of care in AML in vivo model. Single dose of Compound A in combination with daily dosing of venetoclax achieves sustained tumor regression in MOLM-13 xenograft model whereas cytarabine or combination of cytarabine and venetoclax demonstrates no significant anti-tumor activity.

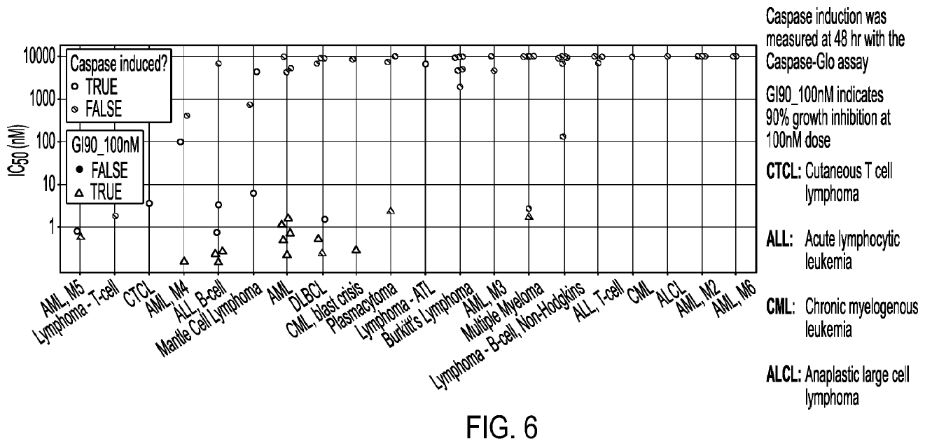

FIG. 6 shows that Compound A is active across multiple heme indications in vitro with AML, T cell lymphomas, mantle cell lymphoma, and DLBCL being the most sensitive.

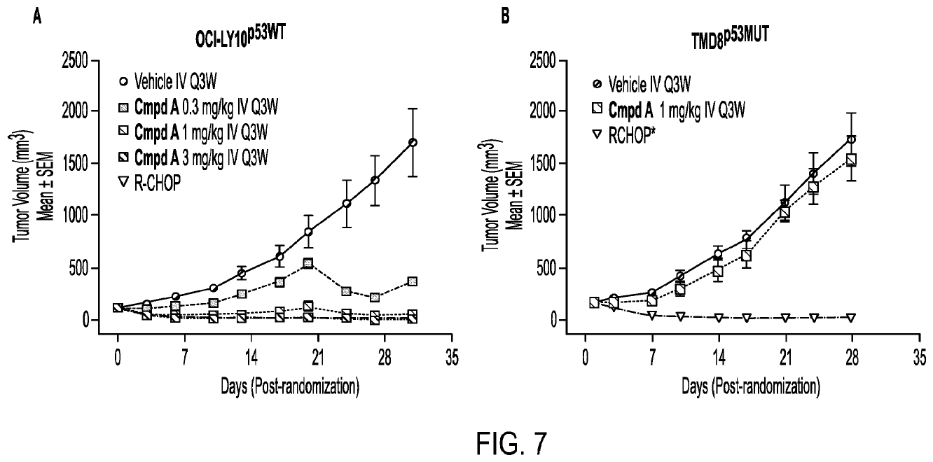

FIG. 7 shows that Compound A is highly active in p53^{WT} ABC-subtype DLBCL. Compound A was highly active in OCI-LY10 p53^{WT} ABC-subtype DLBCL xenograft model (A) but not TMD8 p53^{MUT} ABC-subtype DLBCL xenograft model (B).

In summary of FIGS. 3-7, Compound A dosed intermittently is highly active resulting in responses and complete regression in AML PDX xenograft models. Compound A shows combinatorial benefit with SoC agents in AML in-vitro and in-vivo model, suggesting that Compound A combination can be used for larger patient population. Preclinical data suggest potential for Compound A to be active in additional hematological malignancies, such as DLBCL.

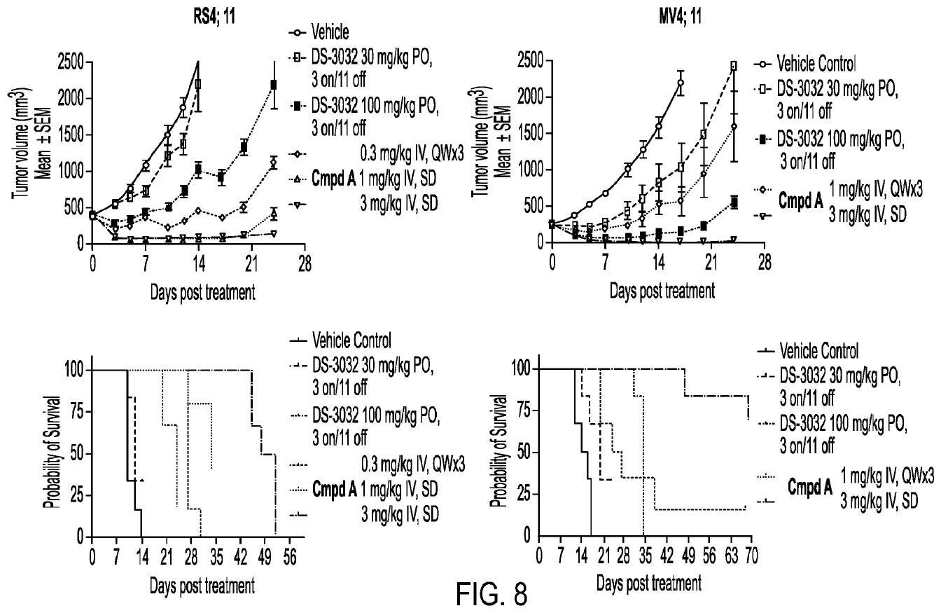

In the RS4;11 ALL model of FIG. 8, the median survival after a single dose of Compound A at 3 mg/kg was 50 days vs. 12 days for the clinically equivalent dosing regimen of DS-3032. In the MV4;11 AML model of FIG. 8, a single dose of Compound A at 3 mg/kg led to complete responses in 5 of 6 animals, and 4 of 6 remain tumor-free on study 80 days post dosing. No complete responses were observed following treatment with DS-3032. n=6 animals/group.

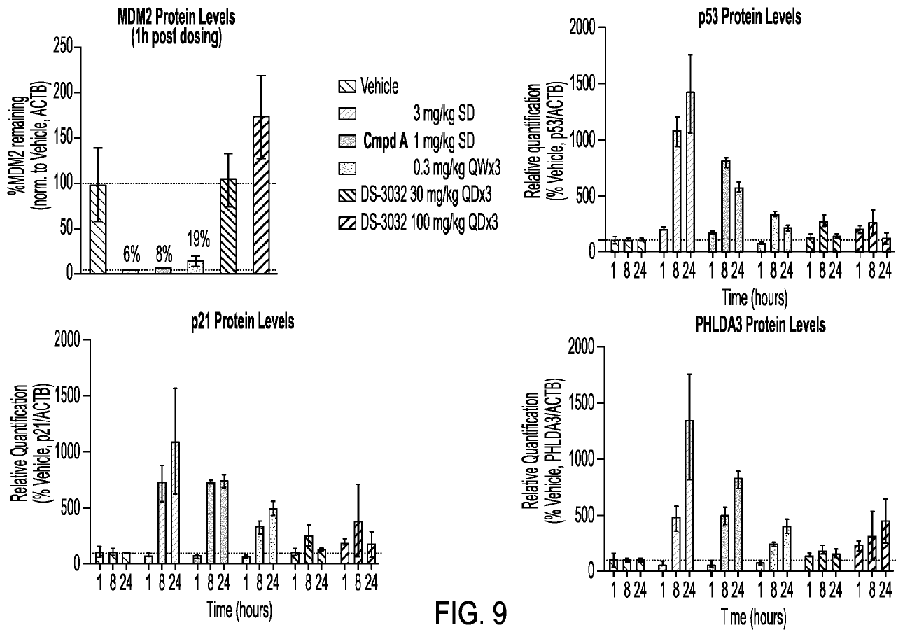

FIG. 9 shows that targeted proteomic analysis of RS4;11 tumors demonstrates robust degradation of MDM2 one hour post dosing. This is associated with activation of the p53 pathway as evidenced by a corresponding upregulation of proteomics biomarkers p53, p21 (cell cycle arrest marker) and PHLDA3 (apoptotic marker).

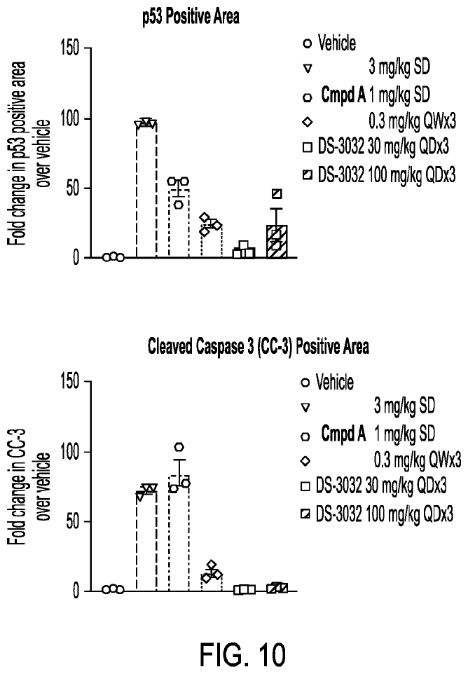

FIG. 10 shows that IHC analysis of RS4;11 tumors demonstrates more robust activation of the p53 pathway and induction of cleaved caspase-3 (CC-3) following a single dose of Compound A than following exposure-matched weekly dosing. Induction of CC-3 was not observed following treatment with the SMI DS-3032 (n=3/group).

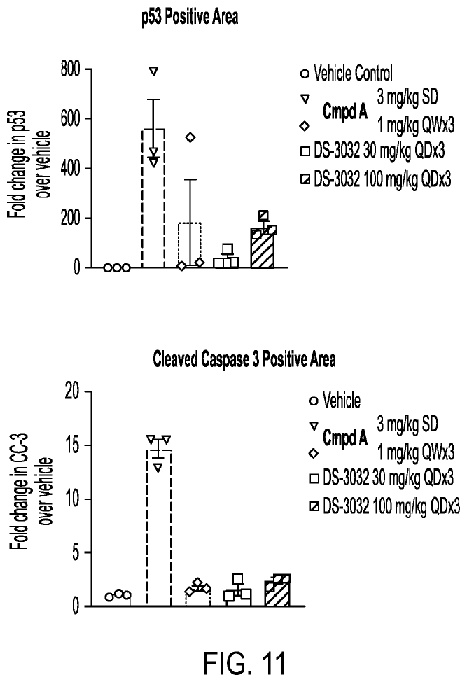

FIG. 11 shows that IHC analysis of MV4;11 tumors demonstrates robust activation of the p53 pathway and induction of cleaved caspase-3 (CC-3) following a single dose of Compound A but not following exposure-matched weekly dosing. Induction of CC-3 following treatment with the DS-3032 was modest and similar to the lower weekly dosing regimen of Compound A (n=3/group).

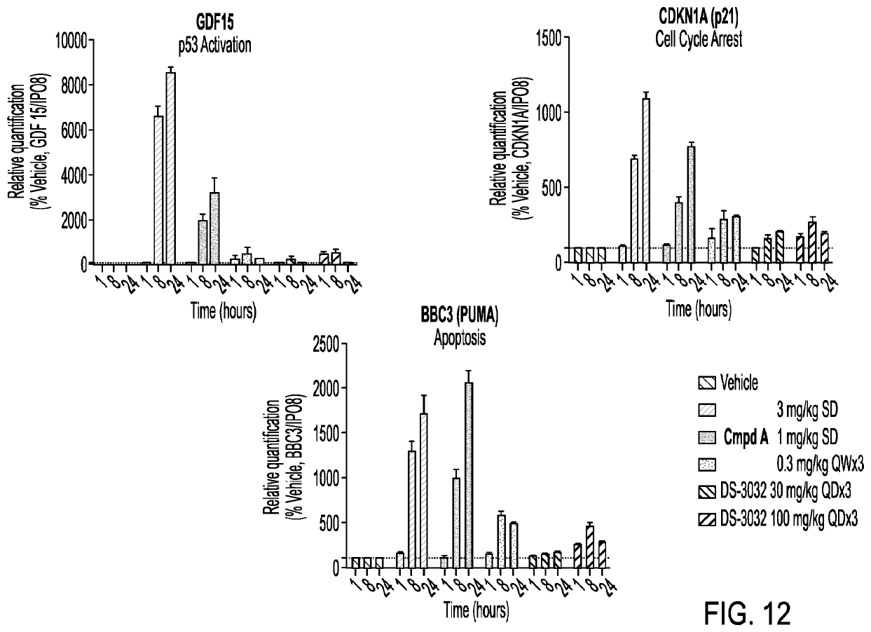

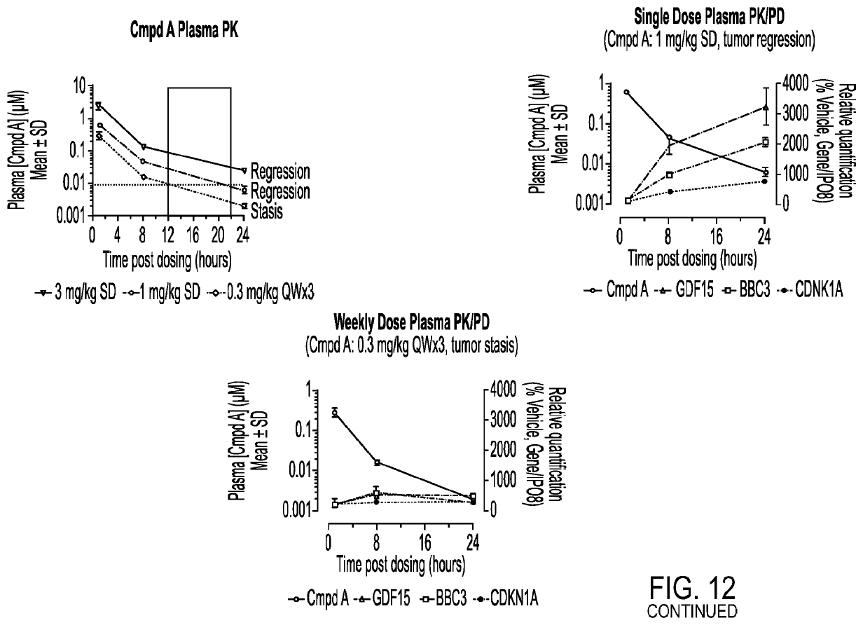

FIG. 12 shows that qPCR analysis of RS4;11 tumors demonstrates robust induction of the apoptotic gene BBC3 following single doses of Compound A that lead to tumor regression (n=3/group). In contrast, exposure-matched weekly dosing of Compound A results in only a moderate increase in apoptotic markers and correlates with tumor stasis (n=3/group). Similar results were observed with the apoptotic genes FAS and DR5 (data not shown). 12 to 22 hours of Compound A plasma concentrations above 0.01 uM are required for tumor regression. Plasma Compound A concentrations above 0.02 uM are required for induction of the apoptotic gene, BBC3.

In summary of FIGS. 8-12, a single high dose of Compound A leads to robust activation of the p53 pathway, apoptosis, and sustained tumor regression, while exposure-matched weekly dosing leads to cell cycle arrest and tumor stasis, as observed with MDM2 small molecule inhibitors. In the MV4;11 AML model, a single dose of Compound A at 3 mg/kg led to complete responses in 5 of 6 animals, and 4 of 6 remain tumor-free on study 80 days post dosing. The pulse dosing regimen of Compound A has the potential to result in improved efficacy and safety profiles compared to the more frequent dosing of MDM2/p53 small molecule inhibitors in the clinic.

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

1. A liquid formulation comprising Compound A, or a pharmaceutically acceptable salt thereof, and a pharmaceutically acceptable excipient and/or carrier, wherein Compound A is (3′R,4′S,5′R)-6″-chloro-4′-(3-chloro-2-fluorophenyl)-N-((1R,4R)-4-(4-(1-(2,6-dioxopiperidin-3-yl)-3-methyl-2-oxo-2,3-dihydro-1H-benzo[d]imidazol-5-yl)piperidine-1-carbonyl)cyclohexyl)-2″-oxodispiro[cyclohexane-1,2′-pyrrolidine-3′,3″-indoline]-5′-carboxamide.

2. The liquid formulation of claim 1, comprising Compound A at a concentration of about 0.05%-5% w/w of the total weight of the formulation.

3. The liquid formulation of claim 1, comprising Compound A at a concentration of about 1-20 mg/mL.

4. The liquid formulation of any one of claims 1-3, comprising a surfactant at a concentration of about 10-30% w/w of the total weight of the formulation.

5. The liquid formulation of any one of claims 1-3, comprising a surfactant at a concentration of about 100-500 mg/mL.

6. The liquid formulation of any one of claims 1-5, comprising one or more carriers at a concentration of about 60-90% w/w of the total weight of the formulation or unit dosage form.

7. The liquid formulation of any one of claims 1-5, comprising a first carrier and a second carrier, wherein the first carrier is a polyol at a concentration of about 60-80% w/w of the total weight of the formulation or unit dosage form and the second carrier is ethanol at a concentration of about 5-20% w/w of the total weight of the formulation or unit dosage form.

8. The liquid formulation of any one of claims 1-7, comprising a first carrier and a second carrier, wherein the first carrier is a polyol and the second carrier is ethanol in a ratio of about 5:1 to about 9:1.

9. The liquid formulation of any one of claims 1-8, which is a unit dosage form.

10. A method for treating a solid cancer or hematological malignancy in a patient, comprising administering to the patient a therapeutically effective amount the liquid formulation of any one of claims 1-9.

11. The method of claim 10, wherein the method comprises administering Compound A to the patient once every three weeks (Q3W).

12. The method of claim 10 or claim 11, wherein the method comprises administering Compound A to the patient by intravenous transfusion.

13. The method of any one of claims 10-12, wherein the solid cancer or hematological malignancy is selected from acute lymphoblastic leukemia (ALL), chronic lymphocytic leukemia (CLL), large granular lymphocytic leukemia (LGL-L), B-cell prolymphocytic leukemia, acute myeloid leukemia (AML), Burkitt lymphoma/leukemia, primary effusion lymphoma, peripheral T-cell lymphoma (PTCL), cutaneous T-cell lymphoma (CTCL), diffuse large B-cell lymphoma (DLBCL), advanced B-cell diffuse large B-cell lymphoma (ABC DLBCL), intravascular large B-cell lymphoma, lymphoplasmacytic lymphoma, Waldenström's macroglobulinemia (WM), splenic marginal zone lymphoma, multiple myeloma, plasmacytoma, uveal melanoma, myelodysplastic syndrome (MDS), or myelodysplastic/myeloproliferative neoplasms (MDS/MPN).

14. The method of any one of claims 10-13, wherein the hematological malignancy is acute lymphoblastic leukemia (ALL) or acute myeloid leukemia (AML).

15. The method of any one of claims 10-14, wherein the solid cancer or hematological malignancy is relapsed and/or refractory.

16. The method of any one of claims 10-15, wherein the patient has received at least two prior therapies.

17. The method of any one of claims 10-16, wherein the patient is a human.

18. The method of any one of claims 10-17, wherein the patient does not have one or more of the exclusion criteria as set forth in Example 2 or 3.

19. The method of any one of claims 10-18, wherein the patient has one or more of the inclusion criteria as set forth in Example 2 or 3.
